(19) United States
(12) Patent Application Publication
(10) Pub. No.: US 2018/0319855 A1
(43) Pub. Date: Nov. 8, 2018

(54) GENETICALLY MUTATED AEQUORIN-BASED BIOSENSORS WITH EXTRA SENSITIVITY TO CALCIUM AND IMPROVED BIOLUMINESCENCE INTENSITY

(71) Applicant: CENTRE NATIONAL DE LA RECHERCHE SCIENTIFIQUE (CNRS), Paris (FR)

(72) Inventors: Adil BAKAYAN, Paris (FR); Nadine PEYRIERAS, Champnetery (FR)

(21) Appl. No.: 15/772,343

(22) PCT Filed: Oct. 28, 2016
(86) PCT No.: PCT/EP2016/076169

§ 371 Date: Apr. 30, 2018

(30) Foreign Application Priority Data
Oct. 30, 2015 (EP) 15306738.4

(51) Int. Cl. C07K 14/435

(52) U.S. Cl. CPC C07K 14/43595; C07K 2319/60

(57) ABSTRACT

Disclosed is an apoaequorin mutant protein, wherein the apoaequorin mutant protein includes an amino acid substitution in position, position, position, position, position or in several of positions and of reference wt-apoaequorin amino acid sequence, as well as a nucleic acid molecule encoding the apoaequorin mutant protein, a recombinant vector, a host cell, a non-human transgenic animal or a transgenic plant including the nucleic acid, uses thereof, a kit including the apoaequorin mutant protein and a reconstituted mutant aequorin complex.

### TECHNICAL FIELD OF THE INVENTION

The present invention is in the field of biochemical assay systems, and more particularly of assay systems for detection of intracellular calcium flux incorporating a mutated biosensor (modified photoproteins), for use both in vitro and in vivo. It relates to mutated apoaequorins having an enhanced affinity for calcium as well as an enhanced bioluminescence and their use as calcium biosensors in cell-based assays and in mouse brain slices.

### BACKGROUND ART

Calcium is known to act as a modulator of many important physiological responses and pathophysiological conditions, participating as a second messenger in the control of a myriad of cellular processes. Accordingly, measurement of intracellular Ca²⁺ concentration is essential in understanding intracellular processes and modulation of cellular proteins. Several Ca²⁺-regulated photoproteins isolated from various organisms, including aequorin, obelin, berovin and mitrocomin, are commonly used for intracellular calcium studies. Among all, the biosensor aequorin is a Ca²⁺ sensor of choice in numerous applications owing to its remarkable flexibility and to the detailed structural and biochemical information available, as well as to its relatively high selectivity and sensitivity to calcium ions, with a broad dynamic range.

Aequorin consists of an apoprotein named apoaequorin (189 amino acids), a noncovalently-bound chromophoric unit (coelenterazine (CLZ)) and molecular oxygen. Apoaequorin contains three calcium-binding sites named EF-hands. Binding of calcium to apoaequorin EF-hands causes the photoprotein to undergo a conformational change, resulting in the oxidation of CLZ (cofactor) with the release of a blue flash of light (465 nm). This process is similar to other photoproteins sensitive to calcium.

Calcium-activated photoproteins, such as aequorin, are being used for detecting calcium flux stimulated by GPCRs, (Stables et al. (1997)), and in drug screening (Ungrin et al. (1999)). In most of these instances, extracellular signals are received through receptors (e.g., G-protein-coupled receptors (GPCRs) and ion channels) and converted to changes in intracellular Ca²⁺ concentration, which results in Ca²⁺ sensitive changes inside the cell, including but not limited to, modulation of Ca²⁺ sensitive kinases, proteases and transcription factors.

However, the light emission from wildtype (wt)-aequorin, as well as from other photoproteins, is characterized by intrinsically low quantum yield that is proportional to its relatively low sensitivity to calcium. In particular, the affinity of aequorin for calcium is rather low (i.e., around 1 μM) relative to the cytosolic concentrations of calcium that are induced by receptors (e.g., in the 0.1 μM to 0.5 μM range). Other calcium-activated photoproteins such as, for example, Obelin, was reported to have lower affinity for calcium and/or low level of light emission (Malikova et al. 2003, Inouye and Sahara, 2007; Bovolenta et al., 2007, Eremeeva et al. 2013a, and Eremeeva et al. 2013b).

Therefore, imaging of typical cytoplasmic changes (few hundred nM upon agonist stimulation) is consequently restricted by the low fractional amount of the emitted photons. In fact, this is the principal problem that significantly limits the spatiotemporal resolution using wt-aequorin.

In order to circumvent this issue, incorporation of different CLZ analogs and genetic mutagenesis have been used to alter calcium sensitivity, decay kinetics, spectral emission and thermostability (Dikici E et al, 2009a).

Studies showed that CLZ analogs tend to be fast and easy way to modify aequorin, but those have different cell membrane permeability and biodistribution (Shimomura, 1997). Besides, only one CLZ analog can be used at the time of multicolor imaging. On a practical basis, increasing aequorin light output is usually achieved by using CLZ analogs other than native (e.g. CLZ-h, CLZ-f and CLZ-hcp), namely, causing higher affinity for calcium.

Similarly, mutagenesis strategy has been used to intrinsically modify apoaequorin at the genetic level, allowing more control, especially in living cells or transgenic organisms where the CLZ analog biodistribution is critical. Genetically modified mutants of apoaequorin with low calcium affinity were reported to be used for measuring calcium in high calcium-containing medium (e.g. endoplasmic reticulum) (Kendall J M, et al 1992, de la Fuente S, et al 2012). However, so far no apoaequorin mutant with increased affinity for calcium has been described.

Hence, what is needed is a new generation of apoeaequorin-based calcium biosensor, displaying high affinity to calcium compared to well-known wt-aequorin, which will significantly enhance the signal-to-noise ratio. Such property is highly desired and would be an essential complementary asset to practically measure minute changes in cellular calcium, which concentration is around several hundreds of nanomolars.

In addition, for monitoring of intracellular calcium levels, fast bioluminescence kinetics are desirable in order to detect fast changes in intracellular calcium levels. As a result, an apoaequorin mutant with increased affinity for calcium should also preferably have at least similar decay kinetics as wt-aequorin, and preferably faster decay kinetics as wt-aequorin.

For cellular or in vivo assays, it is further preferable that the assay may be performed at a temperature acceptable for cells, i.e. close to 37° C. This means that an aequorin mutant with increased affinity for calcium should also preferably be sufficiently thermostable for this purpose, and should thus preferably not have a too much decreased thermostability compared to wt-aequorin. Some aequorin point mutations (e.g. E128G and D153G) have already been reported to increase its thermostability (Tsuzuki et al. 2005). Biosensors, such as aequorin, GA and redquorin, are generally employed to report substrate variation in live cultured cells or in vivo, hence their stability under specific temperatures may affect their performance.

For in vivo imaging, red light-emitting biosensors are highly advantageous, giving the low absorbance and scattering of red wavelengths in living tissues (such as deep tissues and blood cells). Therefore, in recent years several studies focused on red-shifting the emission of wt-aeq from blue to longer wavelengths. Such tuning was accomplished either by site-directed mutagenesis, in case of the mutation Y82F (Stepanyuk et al. 2005), or by fusing aequorin with a fluorescent protein under the principle of bioluminescence resonance energy transfer (BRET). The latter method is preferred due to the incorporation of several properties; such as increased aequorin stability, aequorin being tagged to detectable fluorescent protein and enhanced bioluminescence. The first molecular fusion of aequorin was with GFP (GFP-aeq, or GA) and resulted in shifting aequorin emission to green (509 nm peak) and increased luminescence and protein stability of aequorin (Baubet et al. 2000). GFP is known to be a very stable protein and thus probably stabilizes aequorin. In the same study, the BRET fusion, GA, showed higher sensitivity to calcium by producing almost 20 times higher light signal compared to aequorin alone, in the presence of the same calcium dose. Nowadays, constructing aequorin-based BRET fusions, e.g. aequorin fused to GFP (GA), Venus (VA), Citrine (CitA), or tdTomato (Redquorin), is used for red-shifting aequorin emission (Curie et al. 2007; Bakayan et al. 2011; Bakayan et al. 2015). Nevertheless, the aequorin fusion redquorin does not share such higher affinity for calcium as it is with GA and CitA, for it is similar to wt-aequorin. Consequently, even if redquorin has interestingly different red-shift emission (peak at 582 nm), it lacks the sensitivity to match the signal intensity of GA and CitA.

Hence, the calcium biosensor redquorin is a potential alternative for wt-aequorin (blue wavelength emission). Therefore, a mutant aporedquorin with increased affinity for calcium and emitting a red light would thus be highly desirable for in vivo, in blood cells and also for dual-color applications.

Consequently, there is a need for aequorin mutant proteins that would have, compared to wt-aequorin:
increased affinity for calcium,
increased brightness,
at least similar and preferably faster decay kinetics, and
not much/or significantly reduced and preferably similar thermostability

However, it is well known that mutations improving one functional property may further alter another functional property, and combining several advantageous properties in a single mutant is thus a very difficult goal. For instance, according to Tsuzuki et al. (2005), for all studied aequorin mutants, identified gain of a particular function was associated with loss of other functions that consisted in modification of stability, apparent Ca²⁺ affinity, or decay kinetics. For example mutant presenting mutations Q168R and/or L170I displayed an improved thermostability while the Ca²F affinity was reduced compared to wt-aequorin. Thus, specific aequorin mutants can exhibit different functional modifications which can independently be improved or reduced. These functional modifications cannot be predicted and their mechanism is still unknown.

### SUMMARY OF THE INVENTION

In the context of the present invention, the inventors surprisingly found that apoaequorin with specific directed point-mutation(s) had improved sensitivity to calcium, and therefore increased bioluminescence output. The mutants have at least similar decay kinetics compared to wt-aequorin, some having significantly faster decay kinetics compared to wt-aequorin. Their thermostability and peak emission wavelength were not significantly altered compared to wt-aequorin. In addition, the genetic control of aequorin sensitivity allows its use on other aequorin-based calcium sensors, fusion with a fluorescent protein, with altered emission color (e.g. GFP-aequorin, redquorin . . . ), in particular for two-color calcium measurements.

The present invention thus provides modified forms of apoaequorin, which have an increased affinity for intracellular calcium compared to known photoproteins and in particular compared to wild-type apoaequorin (wt-apoaequorin), and/or exhibit enhanced bioluminescence relative to wt-aequorin, and/or have faster decay kinetics compared to wt-aequorin both in vitro and in cellulo assays.

In a first aspect, the present invention thus relates to an apoaequorin mutant protein comprising an amino acid substitution in position 159, in position 121, in position 123, in position 179, or in position 157, or in several of positions 159, 121, 123, 179 and 157 of reference wt-apoaequorin amino acid sequence (SEQ ID NO:1).

In a second aspect, a nucleic acid molecule is provided which encodes the apoaequorin mutant protein of the invention.

In a third aspect, a recombinant vector is provided, which comprises the nucleic acid molecule encoding the apoaequorin mutant protein of the invention.

Further, the present invention relates to a host cell comprising the nucleic acid molecule or the recombinant vector of the invention.

In another aspect, a non-human transgenic animal or transgenic plant is provided, which comprises the nucleic acid molecule or the recombinant vector of the invention.

In further aspects, the invention relates to the use of the apoaequorin mutant protein, the nucleic acid molecule, the recombinant vector, the host cell, or the non-human transgenic animal or transgenic plant of the invention:
a) for quantitative calcium imaging applications in cell organelles, cells, tissues or non-human animals, using aequorin emission and in particular by bioluminescence resonance energy transfer (BRET);
b) for screening compounds for ability to activate G protein-coupled receptors (GPCRs);
c) in toxicology and genotoxicity tests; or
d) in detection of environmental pollution tests.

Additionally, in further aspects, a kit is provided, which comprises the apoaequorin mutant protein of the invention and a wild-type or modified CLZ cofactor.

In further aspects, a reconstituted mutant aequorin complex is provided, which comprises the apoaequorin mutant protein of the invention, a native or modified synthetic CLZ cofactor, and molecular oxygen.

### DESCRIPTION OF THE FIGURES

FIG. 1. Graphical representation of apoaequorin amino acid structure highlighting the screened mutated sites for enhanced calcium sensitivity. The apoaequorin sequence is 198 amino acids long from Aequorea victoria (PDB code: 1EJ3). The amino acids in squares represent the summary of mutations performed on aequorin protein sequence in order to alter its calcium affinity. CLZ and calcium amino acids contact sites are shown as Clz and Ca, respectively. Calcium-binding EF-hands are displayed as secondary structure in arrows (EF-1, EF-2, EF-3 regions).

FIG. 2. Graphical representation of the results of calcium affinity curves of aequorin mutants proteins. The relationship between calcium concentration ([Ca²⁺]) and fractional bioluminescence intensity (L/Lmax) is displayed in Log values on a linear scale. L and Lmax stand for the peak luminescence intensity at a given [Ca²⁺] and the total luminescence intensity at saturating [Ca²⁺] for the same sample, respectively. Aequorin reconstitution was with CLZ-f. Panel A represents amino acid mutations that resulted in no significant change in affinity, n was at least 2. Panel B shows and categorizes single amino acid mutations that increased or decreased calcium affinity. At pCa 6.5, and relative to Aeq-wt, four groups have been identified with low (e.g. Q140R), medium-low (e.g. S157T), medium-high (e.g. A123D) or high (e.g. Q159D) affinity to calcium, n was at least 3. For Aeq-wt, a sigmoidal curve fit is traced for comparison. R square of the fit goodness was higher than 0.994.

FIG. 3. Graphical representation of the results of calcium affinity curves of aequorin mutant proteins with double mutations. The relationship between calcium concentration ([Ca²⁺]) and fractional bioluminescence intensity (L/Lmax) is displayed in Log values on a linear scale. Aequorin reconstitution was with CLZ-f. The graph shows affinity curves of several combined single mutations that resulted in a group of aequorin mutants with extra-sensitivity, n was at least 4. For Aeq-wt, a sigmoidal curve fit is traced for comparison. R square of the fit goodness was higher than 0.994. The following abbreviations are used for clarity: QD+AT (Q159D+A179T), QD+ND (Q159D+N121D), QD+AD (Q159D+A123D), QT+AT (Q159T+A179T), QT+ND (Q159T+N121D), QT+AD (Q159T+A123D).

FIG. 4. Graphical representation of the results of affinity of calcium affinity curves for redquorin mutant proteins, redquorin-wt (Redq), and GFP-aequorin fusion (GA). The relationship between calcium concentration ([Ca²⁺]) and fractional bioluminescence intensity (L/Lmax) is displayed in Log values on a linear scale. Panels A and B highlight the difference in calcium affinity curves upon using CLZ-native or CLZ-f, respectively (n was at least 4). For Aeq-wt, a sigmoidal curve fit is traced for comparison. R square of the fit goodness was higher than 0.994. The following abbreviations are used for clarity: QD+AT (Q159D+A179T), QT+AT (Q159T+A179T), QD+AD (Q159D+A123D), QT+AD (Q159T+A123D). For more details refer to the legends of FIG. 2 and FIG. 3.

FIG. 5 shows a graphical representation of the results of thermostability for aequorin mutant proteins, GFP-aequorin (GA), citrine-aequorin (CitA), redquorin (Redq) and Redq mutant proteins, at two different temperatures 30 and 40° C. The relative bioluminescence activity at each temperature was calculated by the ratio 2^{nd} counts/1^{st} counts multiplied by 100. The graphical values are mean±SD (error bars), with n between 3 and 5.

FIG. 6. Dose-response curves for activation of endogenous P2Y2 receptor with ATP in CHO cells stably expressing the Ca²⁺ sensor variants. The light intensity was measured by integrating the bioluminescence signal for 30 s, and then normalized for maximum and minimum intensities. Each point is the mean±SD of eight experiments.

FIG. 7. Ca²⁺ imaging of mouse neocortical network with the Redquorin Q159T. The Fluorescence image (Image “Fluo”; left) highlights the expression pattern of the mutant Redquorin in the neocortical slices. The expression was comparable to that of Redquorin WT and GA. Pyramidal neurons expressing the sensors were identified by tdTomato fluorescence (Image “Fluo”; left) where it was preferentially localized in the neocortical layers II/III and V. Imaging started at 25 Hz before perfusing the slices with magnesium (Mg²⁺)-free ACSF. The graph shows bioluminescence intensity related to Ca²⁺ activity starting with spontaneous and neurons-synchronized light flashes. Numbers 1, 2 and their corresponding images display examples of peak bioluminescence intensity of the collective response at these time points. The flashes were ultimately followed with a long lasting cortical spreading depression wave over the entire imaging area (time-point 3 and its corresponding image).

### DETAILED DESCRIPTION OF THE INVENTION

In the context of the present invention, the inventors surprisingly found that apoaequorin with specific directed point-mutation(s) had improved sensitivity to calcium, and therefore increased bioluminescence output. The mutants have at least similar decay kinetics compared to wt-aequorin, some having significantly faster decay kinetics compared to wt-aequorin. Their thermostability and peak emission wavelength were not significantly altered compared to wt-aequorin. In addition, the genetic control of aequorin sensitivity allows its use on other aequorin-based calcium sensors, fusion with a fluorescent protein, with altered emission color (e.g. GFP-aequorin, redquorin . . . ), in particular for two-color calcium measurements.

The present invention thus provides modified forms of apoaequorin, which have an increased affinity for intracellular calcium compared to known photoproteins and in particular compared to wt-apoaequorin, and/or exhibit enhanced bioluminescence relative to wt-aequorin, and/or have faster decay kinetics compared to wt-aequorin both in vitro and in cellulo assays. Aequorin comprising the apoaequorin mutants of the invention represents an essential complementary asset for measuring minute changes in cellular calcium (several hundreds of nanomolars).

Apoaequorin Mutant Protein

In a first aspect, the present invention thus relates to an apoaequorin mutant protein comprising an amino acid substitution in position 159, in position 121, in position 123, in position 179, or in position 157, or in several of positions 159, 121, 123, 179 and 157 of reference wt-apoaequorin amino acid sequence (SEQ ID NO:1).

Wt-apoaequorin refers to the protein having the following amino acid sequence SEQ ID NO:1, with underlined sequences corresponding to EF-hands sequences (EF-Hands I, EF-Hands II and EF-Hands III):

SKLTSDFDNPRWIGRHKHMFNFLDVNHNGKISLDEMVYKASDIVINNLG
ATPEQAKRHKDAVEAFFGGAGMKYGVETDWPAYIEGWKKLATDELEKYA
KNEPTLIRIWGDALFDIVDKDQNGAITLDEWKAYTKAAGIIQSSEDCEE
TFRVCDIDESGQLDVDEMTRQHLGFWYTMDPACEKLYGGAVP

By “apoaequorin mutant protein”, it is meant an apoaequorin protein derived from wt-apoaequorin, which amino acid sequence comprises at least one mutation compared to the amino acid of wt-apoaequorin. By «mutation», it is meant an alteration in the amino acid sequence SEQ ID NO:1 of reference wt-apoaequorin, following modification of the nucleotide sequence encoding said protein. The mutation may be an addition, a deletion or a substitution of an amino acid by another amino acid relative to the original wild-type amino acid sequence. Apoaequorin mutant proteins of the invention comprises an amino acid substitution in position 159, an amino acid substitution in position 121, an amino acid substitution in position 123, an amino acid substitution in position 179, or an amino acid substitution in position 157, or an amino acid substitution in several of positions 159, 121, 123, 179 and 157 of reference wt-apoaequorin amino acid sequence (SEQ ID NO:1). They may optionally further comprise additional mutations (additions, deletions and/or substitutions) compared to wt-apoaequorin amino acid sequence SEQ ID NO:1.

By «amino acid», it is meant, in the context of this invention, that all the residues of the natural α-amino acid (for example alanine (Ala, A), arginine (Arg, R), asparagine (Asn, N), aspartic acid (Asp, D), cysteine (Cys), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y) and valine (Val, V) in the D or L form), as well as non-natural amino acids. Methods allowing introduction of a mutation in a nucleotide sequence are known to the person skilled in the art. For example, it is possible to introduce a mutation by random or directed mutagenesis, by PCR by using degenerate primers, by radiation or by using a mutagenic agent. Said techniques are notably described by Sambrook et al. (2001), and by Ausubel et al. (2011). Preferably, the mutation according to the invention is introduced by site directed mutagenesis. It is understood that in order to introduce said mutations, the skilled person in the art can use functionally equivalent codons (or nucleotide triplets), that is to say codons which code for the same amino acids, or biologically equivalent amino acids. Moreover, should the skilled person in the art wish to optimize the expression of the apoaequorin mutant protein of the invention, s/he can refer to the database on the website http://www.kazusa.or.jp/codon/ which lists the optimal use of codons in various organisms and organelles.

Although further mutations than those determined by the inventors may be present in apoaequorin mutant protein of the invention to increase affinity for calcium, improve brightness, and optionally increase decay kinetics, in order to maintain aequorin bioluminescence activity, the apoaequorin mutant protein of the invention preferably has significant sequence identity with wt-apoaequorin. In particular, the amino acid sequence of the apoaequorin mutant protein of the invention may thus display at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity with reference wt-apoaequorin amino acid sequence SEQ ID NO:1.

The percentage of identity described above can be determined by carrying out an optimal alignment of the amino acid sequences to be compared (here with SEQ ID NO:1 sequence), this percentage being purely statistical and the differences between the two sequences being distributed over their whole length (global alignment). This alignment can be carried out using an algorithm known to the skilled person in the art, such as the global alignment of Needleman and Wunsch (1970) and computerized applications, or just by a mere inspection. The best alignment (that is to say the one producing the highest percentage of identity) is then selected. The percentage of identity between two amino acid sequences is determined by comparing these two sequences globally aligned in an optimal manner, in which the amino acid sequences to be compared can comprise additions or deletions by comparison to the reference sequence (here SEQ ID NO:1 sequence) for an optimal alignment of these two sequences. The percentage of identity is calculated by determining the number of identical positions for which the amino acid residue is identical between the two sequences, dividing this number of identical positions by the total number of positions and multiplying the result obtained by 100 to obtain the percentage of identity between these two sequences.

In order to keep increased affinity for calcium compared to wt-apoaequorin, the amino acid sequence of the apoaequorin mutant protein of the invention should also preferably display significant sequence identity with wt-apoaequorin in the three EF-Hands (calcium binding sites) of apoaequorin.

By “EF-Hands” are meant the three calcium binding sites of apoaequorin, which are located in the reference amino acid sequence of wt-apoaequorin SEQ ID NO:1 at positions 24-35 (EF-Hands I), 117-128 (EF-Hands II), and 153-164 (EF-Hands III). The amino acid sequences and positions of the three EF-Hands of wt-apoaequorin are summarized in Table 1 below.

TABLE 1
Wt-apoaequorin EF-Hands calcium binding sites

| Calcium | Positions in | Amino acid |
|---|---|---|
| binding-site | SEQ ID NO: 1 | sequence |
| EF-Hands I | 24-35 | DVNHNGKISLDE |
| | | (SEQ ID NO: 2) |
| EF-Hands II | 117-128 | DKDQNGAITLDE |
| | | (SEQ ID NO: 3) |
| EF-Hands IIII | 153-164 | DIDESGQLDVDE |
| | | (SEQ ID NO: 4) |

Except for substitutions in positions 121, 123, 159 and/or 157, which are located in EF-Hands, the apoaequorin mutant protein of the invention thus preferably has no or few (preferably no) other mutations in EF-Hands I, II and III compared to wt-apoaequorin. In particular, except for substitutions in positions 121, 123, 159 and/or 157, which are located in EF-Hands, the apoaequorin mutant protein of the invention preferably has at most 3, preferably 0, 1 or 2, more preferably 0 or 1 and most preferably 0 other mutation(s) in EF-Hands I, II and III compared to wt-apoaequorin.

In any case, the aequorin mutant protein of the invention should display increased affinity for calcium compared to wt-aequorin. The term “increased affinity for calcium,” as used herein, refers to any increase in the affinity of an aequorin mutant protein according to the present invention (e.g., apoaequorin set forth in SEQ ID NO:1 having an amino acid substitution at position 159) for calcium relative to wt-aequorin. For example, affinity for calcium may be increased by about 1.5%, or about 2%, or about 3%, or about 3.5%, or about 4%, or about 5%, or about 10%, or about 20%, or about 30%, or about 40%, or about 50%, or about 60%, or about 70%, or about 80%, or about 90%, or about 95%, or about 96%, or about 97%, or about 98%, or about 99% or more, relative to wt-aequorin. In some embodiments, increased affinity for calcium refers to a decrease in the EC50 value of an aequorin mutant protein according to the present invention relative to wt-aequorin. Affinity of a photoprotein for calcium can be measured using well known techniques and assays in the art, including but not limited to, the ones described herein. In an exemplary biochemical assay, described herein, calcium affinity can be measured in vitro by lysing photoprotein-expressing cells, purification of the photoprotein by known methods in the art, then reconstitution with CLZ cofactor (native or modified, such as CLZ-f) followed by measurement of bioluminescence emitted upon the addition of varying concentrations of calcium.

The term “EC50 value for calcium” as used herein, refers to the concentration of free calcium that elicits in vitro a luminescent signal (i.e., bioluminescence) to a level which is 50% of the signal observed for the luminescent signal in the presence of a saturating amount of calcium (i.e., a concentration of calcium above which further increases in calcium concentration do not produce any significant increases in luminescent signal). The EC50 value for calcium, as used herein, is a measure of the affinity of an apoaequorin mutant protein for calcium. The EC50 value can be measured using one or more in vitro assays known in the art, such as biochemical assay described herein.

In some embodiments, the EC50 value for calcium of an aequorin mutant protein according to the invention is decreased by about 1.5%, or 2%, or 3%, or 4%, or 5%, or 10%, or 15%, or 20%, or 25%, or 30%, or 50%, or 60%, or 70%, or 80%, or 90%, or 95%, or greater than 95%, relative to the EC50 value of a wt-aequorin in the same cell. In some embodiments, the EC50 value of an aequorin mutant protein of the present invention is decreased by about 10 nM, or 20 nM, or nM, or 40 nM, or 50 nM, or 60 nM, or 70 nM, or 80 nM, or 90 nM, or 100 nM, or 110 nM, or 120 nM, or 130 nM, or 140 nM, or 150 nM, or 160 nM, or 170 nM, or 180 nM, or 190 nM, or 200 nM, or 210 nM, or 220 nM, or 230 nM, or 240 nM, or 250 nM, or 260 nM, or 270 nM, or 280 nM, or 290 nM, or 300 nM, or 310 nM, or 320 nM, or 330 nM, or 340 nM, or 350 nM, or 360 nM, or 370 nM, or 380 nM, or 390 nM, or 400 nM, or 410 nM, or 420 nM, or 430 nM, or 440 nM, or 450 nM, or decreased by more than 450 nM, relative to the EC50 value of a wt-aequorin

In an exemplary embodiment, an aequorin mutant protein reconstituted with CLZ-f according to the invention preferably has an EC50 value of 492 nM or lower, preferably 216 nM or lower. In another preferred embodiment, an aequorin mutant protein reconstituted with CLZ-native has an EC50 value for calcium of 680 nM or lower, preferably 470 nM or lower.

Preferably, the aequorin mutant protein of the invention displays increased brightness compared to wt-aequorin. The term “increased brightness,” as used herein, refers to any increase in brightness of an aequorin mutant protein relative to wt-photoprotein in the presence of the same Ca²⁺ concentration. For example, in an exemplary embodiment, brightness of a modified aequorin (e.g., apoaequorin having an amino acid modification at position 159) described herein is enhanced relative to the brightness of wt-aequorin, as measured in solutions containing varying concentrations of Ca²⁺. The brightness of an aequorin mutant protein in the presence of calcium may be increased by about 1.5%, or about 2%, or about 3%, or about 4%, or about 5%, or about 10%, or about 20%, or about 30%, or about 40%, or about 50%, or about 60%, or about 70%, or about 80%, or about 90%, or even greater than 90%, relative to that of wt-aequorin. In some embodiments, the bioluminescence of an aequorin mutant protein in the presence of calcium is increased by about 1.5-fold, or 2-fold, or 5-fold, or 10-fold, or 15-fold, or 20-fold, or 25-fold, or 30-fold, or 35-fold, or 40-fold, of 45-fold, or 50-fold, or 55-fold, or 60-fold, or 65-fold, or 70-fold, or 75-fold, or 80-fold, or 85-fold, of 90-fold, or greater than 90-fold, relative to wt-aequorin.

The present invention provides an apoaequorin mutant protein, wherein said apoaequorin mutant protein comprises an amino acid substitution in position 159, position 121, position 123, position 179, or position 157, or in several of positions 159, 121, 123, 179 and 157 of reference wt-apoaequorin amino acid sequence (SEQ ID NO:1).

In a first embodiment, the apoaequorin mutant protein according to the invention comprises an amino acid substitution in position 159 of reference wt-apoaequorin amino acid sequence (SEQ ID NO:1).

Suitable amino acids to replace glutamine (Q) at position 159 of SEQ ID NO:1 include any naturally-occurring or non-naturally occurring amino acid other than lysine (K). In some embodiments, suitable amino acids to replace glutamine at position 159 include negatively-charged amino acids or not positively charged hydrophilic amino acids; preferably one of a naturally occurring amino acid selected from an aspartic acid (D), a glutamic acid (E), an asparagine (N), a glycine (G), a serine (S), a threonine (T), a valine (V), a tyrosine (Y) and a glutamine (Q). In another embodiment, the amino acid to replace glutamine (Q) at position 159 of SEQ ID NO:1 is selected from naturally-occurring or non-naturally occurring amino acids other than positively charged amino acids.

Preferred mutants comprising an amino acid substitution in position 159 of reference wt-apoaequorin amino acid sequence (SEQ ID NO:1) include those in which the glutamine (Q) residue in position 159 of reference wt-apoaequorin amino acid sequence (SEQ ID NO:1) is replaced by an aspartic acid (D) residue (SEQ ID NO:5), a threonine (T) residue (SEQ ID NO:6), a glycine (G) residue (SEQ ID NO:7) or a glutamic acid (E) residue (SEQ ID NO:8). Particularly preferred mutants comprising an amino acid substitution in position 159 of reference wt-apoaequorin amino acid sequence (SEQ ID NO:1) are those in which the glutamine (Q) residue in position 159 of reference wt-apoaequorin amino acid sequence (SEQ ID NO:1) is replaced by an aspartic acid (D) residue (SEQ ID NO:5) or a threonine (T) residue (SEQ ID NO:6).

In another embodiment, the apoaequorin mutant protein according to the invention comprises an amino acid substitution in position 121 of reference wt-apoaequorin amino acid sequence (SEQ ID NO:1).

Suitable amino acids to replace the asparagine (N) residue in position 121 of SEQ ID NO:1 include any naturally-occurring or non-naturally occurring amino acid other than a serine (S). In some embodiments, suitable amino acids to replace asparagine residue in position 121 include one of a naturally occurring amino acid selected from negatively-charged amino acids. A preferred mutant comprising an amino acid substitution in position 121 of reference wt-apoaequorin amino acid sequence (SEQ ID NO:1) is the mutant in which the asparagine (N) residue in position 121 of reference wt-apoaequorin amino acid sequence (SEQ ID NO:1) is replaced by an aspartic acid (D) residue (SEQ ID NO:9). In another embodiment, the amino acid to replace asparagine (N) residue in position 121 of SEQ ID NO:1 is selected from naturally-occurring or non-naturally occurring amino acids other than uncharged hydrophilic amino acids.

By “negatively-charged” it is meant any amino acid with a global negative charge. Among natural amino acids, this includes aspartic acid (D) and glutamic acid (E).

The term “hydrophilic amino acids” refers to any amino acid which is hydrophilic. Hydrophilic amino acids can be negatively-charged (D, E), positively-charged (R, K, H) or uncharged (N, S, T, Q). Examples of such amino acids include aspartate (D), lysine (K), serine (S) and histidine (H)

In another embodiment, the apoaequorin mutant protein according to the invention comprises an amino acid substitution in position 123 of reference wt-apoaequorin amino acid sequence (SEQ ID NO:1).

Suitable amino acids to replace the alanine (A) residue in position 123 of SEQ ID NO:1 include any naturally-occurring or non-naturally occurring amino acid other than serine (S) and threonine (T). In some embodiments, suitable amino acids to replace alanine residue in position 123 include one of a naturally occurring amino acid selected from negatively-charged amino acids. A preferred mutant comprising an amino acid substitution in position 123 of reference wt-apoaequorin amino acid sequence (SEQ ID NO:1) is the mutant in which the alanine (A) residue in position 123 of reference wt-apoaequorin amino acid sequence (SEQ ID NO:1) is replaced by an aspartic acid (D) residue (SEQ ID NO:10). In another embodiment, the amino acid to replace alanine (A) residue in position 123 of SEQ ID NO:1 is selected from naturally-occurring or non-naturally occurring amino acids other than uncharged hydrophilic amino acids.

In another embodiment, the apoaequorin mutant protein according to the invention comprises an amino acid substitution in position 179 of reference wt-apoaequorin amino acid sequence (SEQ ID NO:1).

Suitable amino acids to replace the alanine (A) residue in position 179 of SEQ ID NO:1 include any naturally-occurring or non-naturally occurring amino acid other than the glutamic acid (E) and valine (V). In some embodiments, suitable amino acids to replace alanine residue in position 179 include one of a naturally occurring amino acid selected from hydrophilic amino acids, preferably from not negatively-charged hydrophilic amino acids, more preferably from uncharged hydrophilic amino acids. A preferred mutant comprising an amino acid substitution in position 179 of reference wt-apoaequorin amino acid sequence (SEQ ID NO:1) is the mutant in which the alanine (A) residue in position 179 of reference wt-apoaequorin amino acid sequence (SEQ ID NO:1) is replaced by a threonine (T) residue (SEQ ID NO:11). In another embodiment, the amino acid to replace alanine (A) residue in position 179 of SEQ ID NO:1 is selected from naturally-occurring or non-naturally occurring amino acids other than hydrophobic amino acids and negatively-charged amino acids.

In another embodiment, the apoaequorin mutant protein according to the invention comprises an amino acid substitution in position 157 of reference wt-apoaequorin amino acid sequence (SEQ ID NO:1).

Suitable amino acids to replace the serine (S) residue in position 157 of SEQ ID NO:1 include any naturally-occurring or non-naturally occurring amino acid other than threonine (T), lysine (K) and alanine (A). In some embodiments, suitable amino acids to replace serine residue in position 157 include one of a naturally occurring amino acid selected from negatively-charged amino acids. A preferred mutant comprising an amino acid substitution in position 157 of reference wt-apoaequorin amino acid sequence (SEQ ID NO:1) is the mutant in which the serine (S) residue in position 157 of reference wt-apoaequorin amino acid sequence (SEQ ID NO:1) is replaced by an aspartic acid (D) residue (SEQ ID NO:12). In another embodiment, the amino acid to replace serine (S) residue in position 157 of SEQ ID NO:1 is selected from naturally-occurring or non-naturally occurring amino acids other than hydrophobic amino acids, positively-charged and uncharged hydrophilic amino acids.

In another embodiment, the apoaequorin mutant protein according to the invention comprises substitutions in at least two of positions 159, 121, 13, 179, and 157 of reference wt-apoaequorin amino acid sequence (SEQ ID NO:1).

In this case, the apoaequorin mutant protein preferably comprises an amino acid substitution in position 159, and further comprises an amino acid substitution in position 121, position 123, position 179, position 157, or in several of positions 121, 123, 179 and 157 of reference wt-apoaequorin amino acid sequence (SEQ ID NO:1).

In this case, said amino acid substitution in position 159 preferably consist in replacing the glutamine (Q) residue in position 159 by an aspartic acid (D) residue, a threonine (T) residue, a glycine (G) residue or a glutamic acid (E) residue

In one embodiment, the apoaequorin mutant protein according to the present invention comprises an amino acid substitution in position 159 and an amino acid substitution in position 121 of reference wt-apoaequorin amino acid sequence (SEQ ID NO:1). Preferred mutants according to the invention comprising an amino acid substitution in position 159 and an amino acid substitution in position 121 of reference wt-apoaequorin amino acid sequence (SEQ ID NO:1) include those in which:
the glutamine (Q) residue in position 159 of reference wt-apoaequorin amino acid sequence (SEQ ID NO:1) is replaced by an aspartic acid (D) residue and the asparagine (N) residue in position 121 of reference wt-apoaequorin amino acid sequence (SEQ ID NO:1) is replaced by an aspartic acid (D) residue (SEQ ID NO:13), and
the glutamine (Q) residue in position 159 of reference wt-apoaequorin amino acid sequence (SEQ ID NO:1) is replaced by a threonine (T) residue and the asparagine (N) residue in position 121 of reference wt-apoaequorin amino acid sequence (SEQ ID NO:1) is replaced by an aspartic acid (D) residue (SEQ ID NO:14).

In another embodiment, the apoaequorin mutant protein according to the present invention comprises an amino acid substitution in position 159 and an amino acid substitution in position 123 of reference wt-apoaequorin amino acid sequence (SEQ ID NO:1). Preferred mutants according to the invention comprising an amino acid substitution in position 159 and an amino acid substitution in position 123 of reference wt-apoaequorin amino acid sequence (SEQ ID NO:1) include those in which:
the glutamine (Q) residue in position 159 of reference wt-apoaequorin amino acid sequence (SEQ ID NO:1) is replaced by an aspartic acid (D) residue and the alanine (A) residue in position 123 of reference wt-apoaequorin amino acid sequence (SEQ ID NO:1) is replaced by an aspartic acid (D) residue (SEQ ID NO:15), and
the glutamine (Q) residue in position 159 of reference wt-apoaequorin amino acid sequence (SEQ ID NO:1) is replaced by a threonine (T) residue and the alanine (A) residue in position 123 of reference wt-apoaequorin amino acid sequence (SEQ ID NO:1) is replaced by an aspartic acid (D) residue (SEQ ID NO:16).

In another embodiment, the apoaequorin mutant protein according to the present invention comprises an amino acid substitution in position 159 and an amino acid substitution in position 179 of reference wt-apoaequorin amino acid sequence (SEQ ID NO:1). Preferred mutants according to the invention comprising an amino acid substitution in position 159 and an amino acid substitution in position 179 of reference wt-apoaequorin amino acid sequence (SEQ ID NO:1) include those in which:
the glutamine (Q) residue in position 159 of reference wt-apoaequorin amino acid sequence (SEQ ID NO:1) is replaced by an aspartic acid (D) residue and the alanine (A) residue in position 179 of reference wt-apoaequorin amino acid sequence (SEQ ID NO:1) is replaced by a threonine (T) residue (SEQ ID NO:17), and
the glutamine (Q) residue in position 159 of reference wt-apoaequorin amino acid sequence (SEQ ID NO:1) is replaced by a threonine (T) residue and the alanine (A) residue in position 179 of reference wt-apoaequorin amino acid sequence (SEQ ID NO:1) is replaced by a threonine (T) residue (SEQ ID NO:18).

In another embodiment, the apoaequorin mutant protein according to the present invention comprises an amino acid substitution in position 159 and an amino acid substitution in position 157 of reference wt-apoaequorin amino acid sequence (SEQ ID NO:1). Preferred mutants according to the invention comprising an amino acid substitution in position 159 and an amino acid substitution in position 157 of reference wt-apoaequorin amino acid sequence (SEQ ID NO:1) include those in which:
the glutamine (Q) residue in position 159 of reference wt-apoaequorin amino acid sequence (SEQ ID NO:1) is replaced by an aspartic acid (D) residue and the serine (S) residue in position 157 of reference wt-apoaequorin amino acid sequence (SEQ ID NO:1) is replaced by an aspartic acid (D) residue (SEQ ID NO:19),
the glutamine (Q) residue in position 159 of reference wt-apoaequorin amino acid sequence (SEQ ID NO:1) is replaced by a threonine (T) residue and the serine (S) residue in position 157 of reference wt-apoaequorin amino acid sequence (SEQ ID NO:1) is replaced by an aspartic acid (D) residue (SEQ ID NO:20),

In another embodiment, the apoaequorin mutant protein according to the present invention comprises an amino acid substitution in position 121, and further comprises an amino acid substitution in position 123, position 159, position 179, position 157, or in several of positions 123, 159, 179 and 157 of reference wt-apoaequorin amino acid sequence (SEQ ID NO:1).

In an embodiment, the apoaequorin mutant protein according to the present invention comprises an amino acid substitution in position 121 and an amino acid substitution in position 123 of reference wt-apoaequorin amino acid sequence (SEQ ID NO:1).

In another embodiment, the apoaequorin mutant protein according to the present invention comprises an amino acid substitution in position 121 and an amino acid substitution in position 179 of reference wt-apoaequorin amino acid sequence (SEQ ID NO:1).

In another embodiment, the apoaequorin mutant protein according to the present invention comprises an amino acid substitution in position 121 and an amino acid substitution in position 157 of reference wt-apoaequorin amino acid sequence (SEQ ID NO:1).

In another embodiment, the apoaequorin mutant protein according to the present invention comprises an amino acid substitution in position 121 and an amino acid substitution in position 159 of reference wt-apoaequorin amino acid sequence (SEQ ID NO:1).

In another embodiment, the apoaequorin mutant protein according to the present invention comprises an amino acid substitution in position 123, and further comprises an amino acid substitution in position 121, position 159, position 179, position 157, or in several of positions 121, 159, 179 and 157 of reference wt-apoaequorin amino acid sequence (SEQ ID NO:1).

In an embodiment, the apoaequorin mutant protein according to the present invention comprises an amino acid substitution in position 123 and an amino acid substitution in position 121 of reference wt-apoaequorin amino acid sequence (SEQ ID NO:1).

In another embodiment, the apoaequorin mutant protein according to the present invention comprises an amino acid substitution in position 123 and an amino acid substitution in position 179 of reference wt-apoaequorin amino acid sequence (SEQ ID NO:1).

In another embodiment, the apoaequorin mutant protein according to the present invention comprises an amino acid substitution in position 123 and an amino acid substitution in position 157 of reference wt-apoaequorin amino acid sequence (SEQ ID NO:1).

In another embodiment, the apoaequorin mutant protein according to the present invention comprises an amino acid substitution in position 123 and an amino acid substitution in position 159 of reference wt-apoaequorin amino acid sequence (SEQ ID NO:1).

In another embodiment, the apoaequorin mutant protein according to the present invention comprises an amino acid substitution in position 179, and further comprises an amino acid substitution in position 121, position 123, position 159, position 157, or in several of positions 121, 123, 159 and 157 of reference wt-apoaequorin amino acid sequence (SEQ ID NO:1).

In an embodiment, the apoaequorin mutant protein according to the present invention comprises an amino acid substitution in position 179 and an amino acid substitution in position 121 of reference wt-apoaequorin amino acid sequence (SEQ ID NO:1).

In another embodiment, the apoaequorin mutant protein according to the present invention comprises an amino acid substitution in position 179 and an amino acid substitution in position 123 of reference wt-apoaequorin amino acid sequence (SEQ ID NO:1).

In another embodiment, the apoaequorin mutant protein according to the present invention comprises an amino acid substitution in position 179 and an amino acid substitution in position 157 of reference wt-apoaequorin amino acid sequence (SEQ ID NO:1).

In another embodiment, the apoaequorin mutant protein according to the present invention comprises an amino acid substitution in position 179 and an amino acid substitution in position 159 of reference wt-apoaequorin amino acid sequence (SEQ ID NO:1).

In another embodiment, the apoaequorin mutant protein according to the present invention comprises an amino acid substitution in position 157, and further comprises an amino acid substitution in position 121, position 123, position 179, position 159, or in several of positions 121, 153, 179 and 159 of reference wt-apoaequorin amino acid sequence (SEQ ID NO:1).

In an embodiment, the apoaequorin mutant protein according to the present invention comprises an amino acid substitution in position 157 and an amino acid substitution in position 121 of reference wt-apoaequorin amino acid sequence (SEQ ID NO:1).

In another embodiment, the apoaequorin mutant protein according to the present invention comprises an amino acid substitution in position 157 and an amino acid substitution in position 123 of reference wt-apoaequorin amino acid sequence (SEQ ID NO:1).

In another embodiment, the apoaequorin mutant protein according to the present invention comprises an amino acid substitution in position 157 and an amino acid substitution in position 179 of reference wt-apoaequorin amino acid sequence (SEQ ID NO:1).

In another embodiment, the apoaequorin mutant protein according to the present invention comprises an amino acid substitution in position 157 and an amino acid substitution in position 159 of reference wt-apoaequorin amino acid sequence (SEQ ID NO:1).

In some embodiments, the apoaequorin mutant protein according to the invention comprises additional mutations compared to reference wt-apoaequorin amino acid sequence (SEQ ID NO:1). In particular said additional mutations may be selected from:
a) fusion with a fluorescent protein; and
b) amino acid substitutions that:
   i) increase relative brightness of aequorin,
   ii) increase decay kinetics of aequorin,
   iii) increase thermostability of aequorin, and/or
   iv) increase the wavelength of emission λ of aequorin.

By «fusion with a fluorescent protein», it is meant herein that the apoaequorin mutant protein according to the invention comprises a fluorescent protein at its N- or C-terminal positions, preferably at the N-terminal.

The fusion apoaequorin mutant protein of the present invention may comprise a linker. The term “linker” refers herein to compounds of any kind that are suited to link the said fluorescent protein and the apoaequorin mutant protein. Linkers are preferably not cleaved in the body. Examples of such linkers are SGGSGS and derivatives thereof.

Examples of fluorescent proteins which can be fused in this way to the apoaequorin mutant protein of the invention includes, without limitation, green fluorescent proteins such as GFP, EGFP and derivatives thereof; yellow fluorescent proteins such as YFP, Topaz, EYFP, YPET, SYFP2, Citrine, Venus, cp-Venus and derivatives thereof; fluorescent orange proteins such as Kusabira Orange, Kusabira Orange2, mOrange, mOrange2, dTomato, dTomato-Tandem, DsRed and its variants (DsRed2, DsRed-Express (T1), DsRed-Express2, DsRed-Max, DsRed-Monomer), TagRFP and TagRFP-T and derivatives thereof; red fluorescent proteins such as mRuby, mApple, mStrawberry, AsRed2, mRFP1, JRed, mCherry, eqFP611, tdRFP611, HcRed1, mRaspberry and derivatives thereof; as well as fluorescent proteins emitting in the far red such as tdRFP639, mKate, mKate2, Katushka, tdKatushka, HcRed-Tandem, mPlum and AQ143 (Day et al., 2009) and derivatives thereof. Preferably, the fluorescent protein of the invention is selected among GFP, citrine protein, or tdTomato protein as described above.

Fusions of apoaequorin with GFP, citrine and tdTomato that maintain bioluminescence activity have been described in WO2014016455, WO2001092300 and WO2008104830. In the context of the invention, substitutions disclosed above for wt-aequorin may be inserted into corresponding positions of the fusions to obtain mutated fusions combining the advantages of the apoaequorin mutant proteins according to the invention and the wavelength properties of the fusions.

In this respect, particularly preferred fusions according to the invention are mutated aporedquorin comprising substitutions according to the invention as described above, and notably a aporedquorin mutant of sequence selected from SEQ ID NO:21 to SEQ ID NO:25.

The apoaequorin mutant protein according to the invention may also or alternatively further contain amino acid substitutions that:
i) increase relative brightness of aequorin,
ii) increase decay kinetics of aequorin,
iii) increase thermostability of aequorin, and/or
iv) increase the wavelength of emission A of aequorin.

Suitable “amino acid substitutions that increase relative brightness of aequorin” include the replacement of glutamic acid (E) at position 34 by glycine (G) (E34G), and replacement of valine (V) at position 44 by alanine (A) (V44A). In an exemplary embodiment, these aequorin mutant proteins showed at most 1.9 times more relative light intensity, compared to wt-aequorin protein (Tricoire et al. 2006).

According to the present invention, the terms “increase decay kinetics” refer to the increase in the rate of the emission decay with time, wherein decay rate correspond to the time at which the number of emitted λ photons has reached half-maximum. Suitable “amino acid substitutions that increase decay kinetics of aequorin” include the replacement of methionine (M) in position 19 by cysteine (C) (M19C) and the replacement of phenylalanine (F) at position 113 by tyrosine (Y) (F113Y). In an exemplary embodiment, these aequorin mutant proteins showed at most 1.4 times faster decay rate compared to wt-aequorin protein (Dikici et al. 2009b).

Herein, “increased thermostability” relates to enhanced ability of the mutant protein to retain function after exposure to elevated temperatures compared to the corresponding wild-type protein. Thermostability of aequorin mutant proteins can be measured by calculating the total emitted light recoverable at 40° C. Suitable “amino acid substitutions that increase thermostability of aequorin” include the replacement of glutamic acid (E) at positions 128 by glycine (G) (E128G) and the replacement of aspartic acid (D) at position 153 by glycine (G)(D153G). In an exemplary embodiment, these aequorin mutant proteins showed at most 1.2 times more relative total emitted light at 40° C. compared to wt-aequorin protein (Tsuzuki et al. 2005).

By “increase the wavelength of emission λ”, it is meant that the wavelength of emission A is shifted to a longer wavelength of light, which correspond to a red shift. Suitable “amino acid substitutions that increase the wavelength of emission λ of aequorin” include the replacement of tyrosine (Y) at position 82 by either phenylalanine (F) or tryptophan (W) (Y82F or Y82W). In an exemplary embodiment, these aequorin mutant proteins showed at most 30 nm emission red shift compared to wt-aequorin protein (Stepanyuk et al. 2005).

However, it is understood that the mutant aequorin protein of the present invention should exhibit at least an equivalent Ca²⁺ affinity compared to the wt-aequorin. Preferably, the Ca²⁺ affinity of the mutant aequorin protein of the present invention is improved compared to the wt-aequorin. Those skilled in the art will be able to measure the aequorin Ca²⁺ affinity using routine experimentation.

In various embodiments, the apoaequorin mutant protein according to the invention may also or alternatively include a tag or fusion, either at the N-terminus, C-terminus, or internal region, for detection and/or purification of the apoaequorin mutant protein.

Advantageously, the apoaequorin mutant protein according to the present invention is selected from SEQ ID NO:5 to SEQ ID NO:25 as shown in the Table 2 below.

TABLE 2
Apoaequorin and aporedquorin mutant protein
sequences according to the present invention

| SEQ ID NO: | Base protein | Mutation | Sequence |
|---|---|---|---|
| SEQ ID NO: 1 | apoaequorin | wt | SKLTSDFDNPRWIGRHKHMFNFLDVNHNGKISLDEMVYKASDIVINNLGAT |
| | | | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDESGQLDVDEMTRQHLGFWYTMDPACEKLYGGAVP |
| SEQ ID NO: 5 | apoaequorin | Q159D | SKLTSDFDNPRWIGRHKHMFNFLDVNHNGKISLDEMVYKASDIVINNLGAT |
| | | | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDESGDLDVDEMTRQHLGFWYTMDPACEKLYGGAVP |
| SEQ ID NO: 6 | apoaequorin | Q159T | SKLTSDFDNPRWIGRHKHMFNFLDVNHNGKISLDEMVYKASDIVINNLGAT |
| | | | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDESGTLDVDEMTRQHLGFWYTMDPACEKLYGGAVP |
| SEQ ID NO: 7 | apoaequorin | Q159G | SKLTSDFDNPRWIGRHKHMFNFLDVNHNGKISLDEMVYKASDIVINNLGAT |
| | | | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDESGGLDVDEMTRQHLGFWYTMDPACEKLYGGAVP |
| SEQ ID NO: 8 | apoaequorin | Q159E | SKLTSDFDNPRWIGRHKHMFNFLDVNHNGKISLDEMVYKASDIVINNLGAT |
| | | | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDESGELDVDEMTRQHLGFWYTMDPACEKLYGGAVP |
| SEQ ID NO: 9 | apoaequorin | N121D | SKLTSDFDNPRWIGRHKHMFNFLDVNHNGKISLDEMVYKASDIVINNLGAT |
| | | | PEQAKRHKDAVEAFFGGAGMKYGVETDWPAYIEGWKKLATDELEKYAKNEP |
| | | | TLIRIWGDALFDIVDKDQDGAITLDEWKAYTKAAGIIQSSEDCEETFRVCD |
| | | | IDESGQLDVDEMTRQHLGFWYTMDPACEKLYGGAVP |
| SEQ ID NO: 10 | apoaequorin | A123D | SKLTSDFDNPRWIGRHKHMFNFLDVNHNGKISLDEMVYKASDIVINNLGAT |
| | | | PEQAKRHKDAVEAFFGGAGMKYGVETDWPAYIEGWKKLATDELEKYAKNEP |
| | | | TLIRIWGDALFDIVDKDQNGDITLDEWKAYTKAAGIIQSSEDCEETFRVCD |
| | | | IDESGQLDVDEMTRQHLGFWYTMDPACEKLYGGAVP |
| SEQ ID NO: 11 | apoaequorin | A179T | SKLTSDFDNPRWIGRHKHMFNFLDVNHNGKISLDEMVYKASDIVINNLGAT |
| | | | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDESGQLDVDEMTRQHLGFWYTMDPTCEKLYGGAVP |
| SEQ ID NO: 12 | apoaequorin | S157D | SKLTSDFDNPRWIGRHKHMFNFLDVNHNGKISLDEMVYKASDIVINNLGAT |
| | | | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDEDGQLDVDEMTRQHLGFWYTMDPACEKLYGGAVP |
| SEQ ID NO: 13 | apoaequorin | Q159D + | SKLTSDFDNPRWIGRHKHMFNFLDVNHNGKISLDEMVYKASDIVINNLGAT |
| | | N121D | PEQAKRHKDAVEAFFGGAGMKYGVETDWPAYIEGWKKLATDELEKYAKNEP |
| | | | TLIRIWGDALFDIVDKDQDGAITLDEWKAYTKAAGIIQSSEDCEETFRVCD |
| | | | IDESGDLDVDEMTRQHLGFWYTMDPACEKLYGGAVP |
| SEQ ID NO: 14 | apoaequorin | Q159T + | SKLTSDFDNPRWIGRHKHMFNFLDVNHNGKISLDEMVYKASDIVINNLGAT |
| | | N121D | PEQAKRHKDAVEAFFGGAGMKYGVETDWPAYIEGWKKLATDELEKYAKNEP |
| | | | TLIRIWGDALFDIVDKDQDGAITLDEWKAYTKAAGIIQSSEDCEETFRVCD |
| | | | IDESGTLDVDEMTRQHLGFWYTMDPACEKLYGGAVP |
| SEQ ID NO: 15 | apoaequorin | Q159D + | SKLTSDFDNPRWIGRHKHMFNFLDVNHNGKISLDEMVYKASDIVINNLGAT |
| | | A123D | PEQAKRHKDAVEAFFGGAGMKYGVETDWPAYIEGWKKLATDELEKYAKNEP |
| | | | TLIRIWGDALFDIVDKDQNGDITLDEWKAYTKAAGIIQSSEDCEETFRVCD |
| | | | IDESGDLDVDEMTRQHLGFWYTMDPACEKLYGGAVP |
| SEQ ID NO: 16 | apoaequorin | Q159T + | SKLTSDFDNPRWIGRHKHMFNFLDVNHNGKISLDEMVYKASDIVINNLGAT |
| | | A123D | PEQAKRHKDAVEAFFGGAGMKYGVETDWPAYIEGWKKLATDELEKYAKNEP |
| | | | TLIRIWGDALFDIVDKDQNGDITLDEWKAYTKAAGIIQSSEDCEETFRVCD |
| | | | IDESGTLDVDEMTRQHLGFWYTMDPACEKLYGGAVP |
| SEQ ID NO: 17 | apoaequorin | Q159D + | SKLTSDFDNPRWIGRHKHMFNFLDVNHNGKISLDEMVYKASDIVINNLGAT |
| | | A179T | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDESGDLDVDEMTRQHLGFWYTMDPTCEKLYGGAVP |
| SEQ ID NO: 18 | apoaequorin | Q159T + | SKLTSDFDNPRWIGRHKHMFNFLDVNHNGKISLDEMVYKASDIVINNLGAT |
| | | A179T | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDESGTLDVDEMTRQHLGFWYTMDPTCEKLYGGAVP |
| SEQ ID NO: 19 | apoaequorin | Q159D + | SKLTSDFDNPRWIGRHKHMFNFLDVNHNGKISLDEMVYKASDIVINNLGAT |
| | | S157D | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDEDGDLDVDEMTRQHLGFWYTMDPACEKLYGGAVP |
| SEQ ID NO: 20 | apoaequorin | Q159T + | SKLTSDFDNPRWIGRHKHMFNFLDVNHNGKISLDEMVYKASDIVINNLGAT |
| | | S157D | PEQAKRHKDAVEAFFGGAGMKYGVETDWPAYIEGWKKLATDELEKYAKNEP |
| | | | TLIRIWGDALFDIVDKDQNGAITLDEWKAYTKAAGIIQSSEDCEETFRVCD |
| | | | IDEDGTLDVDEMTRQHLGFWYTMDPACEKLYGGAVP |
| SEQ ID NO: 21 | aporedquorin | Q159D | MVSKGEEVIKEFMRFKVRMEGSMNGHEFEIEGEGEGRPYEGTQTAKLKVTK |
| | | | GGPLPFAWDILSPQFMYGSKAYVKHPADIPDYKKLSFPEGFKWERVMNFED |
| | | | GGLVTVTQDSSLQDGTLIYKVKMRGTNFPPDGPVMQKKTMGWEASTERLYP |
| | | | RDGVLKGEIHQALKLKDGGHYLVEFKTIYMAKKPVQLPGYYYVDTKLDITS |
| | | | HNEDYTIVEQYERSEGRHHLFLGHGTGSTGSGSSGTASSEDNNMAVIKEFM |
| | | | RFKVRMEGSMNGHEFEIEGEGEGRPYEGTQTAKLKVTKGGPLPFAWDILSP |
| | | | QFMYGSKAYVKHPADIPDYKKLSFPEGFKWERVMNFEDGGLVTVTQDSSLQ |
| | | | DGTLIYKVKMRGTNFPPDGPVMQKKTMGWEASTERLYPRDGVLKGEIHQAL |
| | | | KLKDGGHYLVEFKTIYMAKKPVQLPGYYYVDTKLDITSHNEDYTIVEQYER |
| | | | SEGRHHLFLSGFDNPRWIGRHKHMFNFLDVNHNGKISLDEMVYKASDIVIN |
| | | | NLGATPEQAKRHKDAVEAFFGGAGMKYGVETDWPAYIEGWKKLATDELEKY |
| | | | AKNEPTLIRIWGDALFDIVDKDQNGAITLDEWKAYTKAAGIIQSSEDCEET |
| | | | FRVCDIDESGDLDVDEMTRQHLGFWYTMDPACEKLYGGAVP |
| SEQ ID NO: 22 | aporedquorin | Q159T | MVSKGEEVIKEFMRFKVRMEGSMNGHEFEIEGEGEGRPYEGTQTAKLKVTK |
| | | | GGPLPFAWDILSPQFMYGSKAYVKHPADIPDYKKLSFPEGFKWERVMNFED |
| | | | GGLVTVTQDSSLQDGTLIYKVKMRGTNFPPDGPVMQKKTMGWEASTERLYP |
| | | | RDGVLKGEIHQALKLKDGGHYLVEFKTIYMAKKPVQLPGYYYVDTKLDITS |
| | | | HNEDYTIVEQYERSEGRHHLFLGHGTGSTGSGSSGTASSEDNNMAVIKEFM |
| | | | RFKVRMEGSMNGHEFEIEGEGEGRPYEGTQTAKLKVTKGGPLPFAWDILSP |
| | | | QFMYGSKAYVKHPADIPDYKKLSFPEGFKWERVMNFEDGGLVTVTQDSSLQ |
| | | | DGTLIYKVKMRGTNFPPDGPVMQKKTMGWEASTERLYPRDGVLKGEIHQAL |
| | | | KLKDGGHYLVEFKTIYMAKKPVQLPGYYYVDTKLDITSHNEDYTIVEQYER |
| | | | SEGRHHLFLSGFDNPRWIGRHKHMFNFLDVNHNGKISLDEMVYKASDIVIN |
| | | | NLGATPEQAKRHKDAVEAFFGGAGMKYGVETDWPAYIEGWKKLATDELEKY |
| | | | AKNEPTLIRIWGDALFDIVDKDQNGAITLDEWKAYTKAAGIIQSSEDCEET |
| | | | FRVCDIDESGTLDVDEMTRQHLGFWYTMDPACEKLYGGAVP |
| SEQ ID NO: 23 | aporedquorin | Q159D + | MVSKGEEVIKEFMRFKVRMEGSMNGHEFEIEGEGEGRPYEGTQTAKLKVTK |
| | | A179T | GGPLPFAWDILSPQFMYGSKAYVKHPADIPDYKKLSFPEGFKWERVMNFED |
| | | | GGLVTVTQDSSLQDGTLIYKVKMRGTNFPPDGPVMQKKTMGWEASTERLYP |
| | | | RDGVLKGEIHQALKLKDGGHYLVEFKTIYMAKKPVQLPGYYYVDTKLDITS |
| | | | HNEDYTIVEQYERSEGRHHLFLGHGTGSTGSGSSGTASSEDNNMAVIKEFM |
| | | | RFKVRMEGSMNGHEFEIEGEGEGRPYEGTQTAKLKVTKGGPLPFAWDILSP |
| | | | QFMYGSKAYVKHPADIPDYKKLSFPEGFKWERVMNFEDGGLVTVTQDSSLQ |
| | | | DGTLIYKVKMRGTNFPPDGPVMQKKTMGWEASTERLYPRDGVLKGEIHQAL |
| | | | KLKDGGHYLVEFKTIYMAKKPVQLPGYYYVDTKLDITSHNEDYTIVEQYER |
| | | | SEGRHHLFLSGFDNPRWIGRHKHMFNFLDVNHNGKISLDEMVYKASDIVIN |
| | | | NLGATPEQAKRHKDAVEAFFGGAGMKYGVETDWPAYIEGWKKLATDELEKY |
| | | | AKNEPTLIRIWGDALFDIVDKDQNGAITLDEWKAYTKAAGIIQSSEDCEET |
| | | | FRVCDIDESGDLDVDEMTRQHLGFWYTMDPTCEKLYGGAVP |
| SEQ ID NO: 24 | aporedquorin | Q159T + | MVSKGEEVIKEFMRFKVRMEGSMNGHEFEIEGEGEGRPYEGTQTAKLKVTK |
| | | A179T | GGPLPFAWDILSPQFMYGSKAYVKHPADIPDYKKLSFPEGFKWERVMNFED |
| | | | GGLVTVTQDSSLQDGTLIYKVKMRGTNFPPDGPVMQKKTMGWEASTERLYP |
| | | | RDGVLKGEIHQALKLKDGGHYLVEFKTIYMAKKPVQLPGYYYVDTKLDITS |
| | | | HNEDYTIVEQYERSEGRHHLFLGHGTGSTGSGSSGTASSEDNNMAVIKEFM |
| | | | RFKVRMEGSMNGHEFEIEGEGEGRPYEGTQTAKLKVTKGGPLPFAWDILSP |
| | | | QFMYGSKAYVKHPADIPDYKKLSFPEGFKWERVMNFEDGGLVTVTQDSSLQ |
| | | | DEGTLIYKVKMRGTNFPPDGPVMQKKTMGWEASTERLYPRDGVLKGIHQAL |
| | | | KLKDGGHYLVEFKTIYMAKKPVQLPGYYYVDTKLDITSHNEDYTIVEQYER |
| | | | SEGRHHLFLSGFDNPRWIGRHKHMFNFLDVNHNGKISLDEMVYKASDIVIN |
| | | | NLGATPEQAKRHKDAVEAFFGGAGMKYGVETDWPAYIEGWKKLATDELEKY |
| | | | AKNEPTLIRIWGDALFDIVDKDQNGAITLDEWKAYTKAAGIIQSSEDCEET |
| | | | FRVCDIDESGTLDVDEMTRQHLGFWYTMDPTCEKLYGGAVP |
| SEQ ID NO: 25 | aporedquorin | Q159T + | MVSKGEEVIKEFMRFKVRMEGSMNGHEFEIEGEGEGRPYEGTQTAKLKVTK |
| | | A123D | GGPLPFAWDILSPQFMYGSKAYVKHPADIPDYKKLSFPEGFKWERVMNFED |
| | | | GGLVTVTQDSSLQDGTLIYKVKMRGTNFPPDGPVMQKKTMGWEASTERLYP |
| | | | RDGVLKGEIHQALKLKDGGHYLVEFKTIYMAKKPVQLPGYYYVDTKLDITS |
| | | | HNEDYTIVEQYERSEGRHHLFLGHGTGSTGSGSSGTASSEDNNMAVIKEFM |
| | | | RFKVRMEGSMNGHEFEIEGEGEGRPYEGTQTAKLKVTKGGPLPFAWDILSP |
| | | | QFMYGSKAYVKHPADIPDYKKLSFPEGFKWERVMNFEDGGLVTVTQDSSLQ |
| | | | DGTLIYKVKMRGTNFPPDGPVMQKKTMGWEASTERLYPRDGVLKGEIHQAL |
| | | | KLKDGGHYLVEFKTIYMAKKPVQLPGYYYVDTKLDITSHNEDYTIVEQYER |
| | | | SEGRHHLFLSGFDNPRWIGRHKHMFNFLDVNHNGKISLDEMVYKASDIVIN |
| | | | NLGATPEQAKRHKDAVEAFFGGAGMKYGVETDWPAYIEGWKKLATDELEKY |
| | | | AKNEPTLIRIWGDALFDIVDKDQNGDITLDEWKAYTKAAGIIQSSEDCEET |
| | | | FRVCDIDESGTLDVDEMTRQHLGFWYTMDPACEKLYGGAVP |

The apoaequorin mutant protein of the present invention can be prepared using any suitable methods known in the art. For example, standard techniques for site-directed mutagenesis of nucleic acids may be used such as those described, for example, in the laboratory manual entitled Molecular Cloning by Sambrook, Fritsch and Maniatis. Additionally, standard molecular biology techniques involving polymerase chain reaction (PCR) mutagenesis may be used.

In some embodiments, the apoaequorin mutant protein is generated using standard genetic engineering techniques. For example, a nucleic acid molecule encoding a wt-photoprotein or a portion thereof can be cloned into a suitable vector for expression in an appropriate host cell. Suitable expression vectors are well known in the art and typically include the necessary elements for the transcription and translation of the modified photoprotein coding sequence.

Modified apoaequorin mutant protein described herein may also be synthesized chemically from amino acid precursors using methods well known in the art, including solid phase peptide synthetic method.

Fusion of the apoaequorin mutant protein to a fluorescent protein for the construction of the biosensor of the invention is carried out using genetic engineering, enzymatic or chemical coupling techniques known to the skilled person in the art. Such techniques are described in Sambrook et al. (2001) and Ausubel et al. (2011).

In a preferred embodiment, the apoaequorin mutant protein according to the invention is further covalently conjugated to another molecule able to specifically bind a target molecule, preferentially the apoaequorin mutant protein according to the invention is further covalently conjugated to an antibody.

According to the invention said target molecule can either be:
an analyte of interest, which may then be directly detected. The molecule specifically binding the analyte may then be an antibody or a receptor (when the analyte is a ligand of the receptor) or a ligand (when the analyte is a receptor of the ligand); or
a molecule capable to specifically bind a second molecule specifically binding an analyte of interest for an indirect detection. For example the target molecule may be a biotin, in which case the apoaequorin mutant of the invention may be conjugated to avidin. The analyte of interest may then be detected using a biotinylated antibody, receptor or ligand, which is further detected with an apoaequorin mutant-avidin conjugate according of the invention. In another example, the target molecule may be a primary antibody, in which case the apoaequorin mutant of the invention may be conjugated to a secondary antibody specifically binding to the primary antibody. The analyte of interest may then be detected using a primary antibody, which is further detected with an apoaequorin mutant-secondary antibody conjugate according to the invention.

As a result, in a preferred embodiment, the apoaequorin mutant protein according to the invention may be covalently conjugated to an antibody, a receptor, a ligand. In a particularly preferred embodiment, the apoaequorin mutant protein according to the invention may be covalently conjugated to an antibody, a biotin or an avidin.

The conjugated apoaequorin mutant protein with another molecule as defined above can be prepared using any suitable methods known in the art.

Nucleic Acid Molecules, Vectors, Host Cells, Non-Human Transgenic Animals, Transgenic Plants

Also encompassed by the present invention are nucleic acid molecules encoding the apoaequorin mutant protein of the invention and recombinant vector comprising such nucleic acid molecule encoding the apoaequorin mutant protein.

Any nucleic acid molecule encoding the apoaequorin mutant protein of the invention may be used. In particular, due to genetic code degeneracy, a particular apoaequorin mutant protein of the invention may be encoded by many distinct nucleic acid molecules. It should be understood that the said nucleic acid molecules encoding the apoaequorin mutant protein of the invention can be modified by the skilled person in the art using functionally equivalent codons (or nucleotide triplets), that is to say codons which code for the same amino acids, or biologically equivalent amino acids. Moreover, should the skilled person in the art wish to optimize the expression of the apoaequorin mutant protein of the invention, s/he can refer to the database on the website http://www.kazusa.or.jp/codon/ which lists the optimal use of codons in various organisms and organelles.

As used herein, the term “recombinant vector” refers to a vector transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. The recombinant vector may comprise a structural gene and a promoter for regulating expression thereof, and in addition, various regulatory elements in a state that allows them to operate within host cells. It is well known in the art that a type of recombinant vector of a living organism such as an animal and a species of a regulatory element used may vary depending on the type of host cell used.

The term “vector” as used herein refers to “plasmid vector” or “viral vector”.

The term “plasmid” refers to a circular, extrachromosomal nucleic acid molecule capable of replication in a cell, such as pcDNA3, pTriEx, bluescript, etc

The term “viral vector” refers to a nucleic acid vector that includes at least one element of a virus genome and may be packaged into a viral particle (e.g. pSinRep5). The terms “virus”, “virions”, “viral particles” and “viral vector particle” are used interchangeably to refer to viral particles that are formed when the nucleic acid vector is transduced into an appropriate cell or cell line according to suitable conditions allowing the generation of viral particles. In the context of the present invention, the term “viral vector” has to be understood broadly as including nucleic acid vector (e.g. DNA viral vector) as well as viral particles generated thereof. A “viral vector” is used herein according to its art-recognized meaning. It refers to any vector that comprises at least one element of viral origin, including a complete viral genome, a portion thereof or a modified viral genome as described below as well as viral particles generated thereof (e.g. viral vector packaged into a viral capsid to produce infectious viral particles). Viral vectors of the invention can be replication-competent, or can be genetically disabled so as to be replication-defective or replication-impaired. The term “replication-competent” as used herein encompasses replication-selective and conditionally-replicative viral vectors which are engineered to replicate better or selectively in specific host cells (e.g. neuronal and tumoral cells). Viral vectors may be obtained from a variety of different viruses, and especially from a virus selected from the group consisting of retrovirus, adenovirus, adeno-associated virus (AAV), poxvirus, herpes virus, measle virus and foamy virus.

In a preferred embodiment, the said recombinant vector comprise “regulatory elements” or “regulatory sequence” to control the expression of the said nucleic acid molecule encoding the apoaequorin mutant protein

As used herein, the term “regulatory elements” or “regulatory sequence” refers to any element that allows, contributes or modulates the expression of nucleic acid molecule(s) in a given host cell or subject, including replication, duplication, transcription, splicing, translation, stability and/or transport of the nucleic acid(s) or its derivative (i.e. mRNA). The vector may be prepared by conventional methods known in the art, for example, by obtaining an apoaequorin mutant gene having a nucleic acid sequence as disclosed above by PCR amplification and ligating the amplified product into a suitable vector of interest.

The invention also relates to a host cell comprising the nucleic acid molecule according to the invention or the recombinant vector according to the invention.

By «host cell», it is referred herein to a prokaryotic or eukaryotic cell, capable of replicating the nucleic acid coding for the mutated bioluminescent protein according to the invention or the recombinant vector as previously described, and thus capable of expressing the mutated bioluminescent protein of the invention. A host cell can be «transfected» or «transformed» according to a process known to the skilled person in the art by means of which said nucleic acid or said vector is transferred or introduced into the host cell. Examples of such methods include, without limitation, electroporation, lipofection, calcium phosphate transfection, transfection via DEAE-Dextran, microinjection, and biolistic.

Among host cells are included, without limitation, bacteria, yeasts, fungi, or any other higher eukaryotic cell. The skilled person in the art can therefore choose the appropriate host cells among the many available cell lines, notably via the American Type Culture Collection (ATCC) (www.ATCC.org). Examples of host cells include, without limitation, microorganisms such as Gram negative bacteria of the genus Escherichia (for example, E. coli RR1, LE392, B, X 1776, W3110, DH5 alpha, JM109, KC8), Serratia, Pseudomonas, Erwinia, Methylobacterium, Rhodobacter, Salmonella or Zymomonas, Gram positive bacteria of the genus Corynebacterium, Brevibacterium, Bacillus, Arthrobacter, or Streptomyces, yeasts of the Saccharomyces genus, cells from fungi of the genus

Aspergillus, Neurospora, Fusarium and Trichoderma, animal cells including HEK293, NIH3T3, Jurkat, MEF, Vero, HeLa, CHO, W138, BHK, COS-7, MDCK, C127, Saos, PC12, HKG, and insect cells Sf9, Sf21, Hi Five or Bombyx mori. The use of insect cells is described in particular in the manual «Baculovirus Expression vectors, A Laboratory Manual», by David R. O'Reilly et al., Oxford University Press, USA, (1992).

In the case where the host cell is transformed by the recombinant vector of the invention as described above, the choice of said host cell can be dictated by the choice of said vector, and depending on the chosen use, that is to say cloning of the nucleic acid or expression of the mutated cyan fluorescent protein of the invention.

The invention also relates to a non-human transgenic animal or transgenic plant comprises a nucleic acid molecule encoding the apoaequorin mutant protein or the recombinant vector comprising the nucleic acid molecule encoding the apoaequorin mutant protein.

The term “transgenic animal” as used herein refers to a non-human animal with a transgene as described above with a trait changed due to a heterologous recombinant gene integrated into its genome. The examples of non-human animal include vertebrate, in particular mammals, particularly mammals which is able to develop a disease similar to the ones found in human so that one may use it as a model to study etiology and pathogenesis of a disease of interest. The suitable animals include vertebrates having an internal structure, an immune system and/or a body temperature similar to those of humans and being capable of developing a high blood pressure, cancer or immune deficiency. By way of example, vertebrates such as mice, rats, zebrafish, sheep, pigs, goats, camels, antelope, dogs, rabbits, guinea pigs, or hamsters may be used.

A transgenic non-human animal expressing an apoaequorin mutant protein may be prepared by any conventional method known in the art.

As defined herein, a “transgenic plant” is a plant that has been altered using recombinant DNA technology to contain an isolated nucleic acid which would otherwise not be present in the plant.

A transgenic plant expressing an apoaequorin mutant protein may be prepared by any conventional method known in the art.

Uses

The invention also relates to the use of the apoaequorin mutant protein, the nucleic acid molecule, the recombinant vector, the host cell, or the non-human transgenic animal or transgenic plant according to the invention:
a) for quantitative imaging applications in cell organelles, cells, tissues or non-human animals, in particular by bioluminescence resonance energy transfer (BRET);
b) for screening compounds for ability to activate G protein-coupled receptors (GPCRs);
c) in toxicology and genotoxicity tests; or
d) in detection of environmental pollution tests; or
e) for photoprotein-based bioluminescent immunoassays.

The «bioluminescence resonance energy transfer (BRET)”, differs from FRET in that the energy of the «donor» originates from a bioluminescent molecule, such as CLZ, which is excited in the presence of a photoprotein (e.g. aequorin) and emits photons. The resonance energy of these photons is then transferred to an «acceptor» fluorescent molecule (such as GFP, citrine, tdTomato, and fluorescent quantum dots, etc.) which emits light at its corresponding wavelength when the conditions of proximity and geometry for energy transfer are met. The fluorescent molecule according to the invention must therefore be used as an «acceptor» in this type of BRET application.

The term “GPCR” refers to G-protein coupled receptors, which are involved in various cellular signal transduction pathways. As one of the largest and most diverse protein families in nature, the G-protein coupled receptor (GPCR) superfamily plays important roles in a variety of biological and pathological processes such as development and proliferation, neuromodulation, angiogenesis, metabolic disorders, inflammation, and viral infection. It is one of the most targeted protein families in pharmaceutical research today. All members of the GPCR superfamily share a similar seven transmembrane domain, however, can be grouped into classes on the basis of shared sequence motifs. For example, Class A includes Rhodopsin-like GPCRs, Class B includes Secretin-like GPCRs, Class C includes Metabotropic glutamate/pheromone GPCRs, Class D includes fungal pheromone GPCRs, and Class E includes cAMP GPCRs. Additional GPCRs can be classified as Frizzled/Smoothened GPCRs, Vomeronasal GPCRs and some that remain unclassified.

The term “bioluminescent immunoassays” refers to the use of bioluminescence as readout method for immunological bioassays, such as ELISA, immunohistochemistry . . . . Immunoassays are set of bioanalytical tools to detect and quantify an analyte in a sample based on the reaction of the analyte with an antibody. The latter is usually highly specific anti-analyte antibody. An analyte can be either natural or synthetic compound of various forms: like hormones, antibiotics, antiepileptics, antidepressants, psychiatric drugs, vitamins, tumor markers, antigens, antibodies, etc. A sample from which the analyte is detected could be blood, serum, plasma, urine, saliva, milk, animal tissue, plant tissue, food, etc.

Immunoassays can be classified as competitive, non-competitive, heterogeneous and homogenous. They all aim at identifying a label that is measured to estimate the amount of analyte present in a sample. The “label” can mainly be either enzyme or radioactive isotope that produces light or causes color changes, respectively. Photoproteins are type of label that produces bioluminescent light, which can be easily detected and quantified. The unknown analyte concentrations can be determined from calibration curves.

Immunoassays have been extensively applied to important areas such as disease diagnosis, clinical pharmacokinetic studies, therapeutic drug monitoring and drug discovery.

In a preferred embodiment, the present invention is directed to the use of a conjugated apoaequorin mutant protein according to the invention to directly or indirectly detect an analyte of interest by a bioluminescent immunoassays as described above.

The invention also relates to the use of said products in toxicology, genotoxicity or environmental pollution detection tests carried out in solution, more particularly from a sample, a biological extract, a cell, tissue or a living organisms.

The invention also relates to a kit comprises the apoaequorin mutant protein and a wild-type or modified CLZ cofactor.

The term CLZ is defined as a molecule with an imidazopyrazine structure, characterized by its ability to luminesce when bound to a given apophotoprotein or luciferase in solution. CLZ are known to luminesce when excitation reaction is triggered by a wide variety of luminogenic proteins, specifically marine photoproteins (e.g. aequorin, obelin, berovin etc.) and luciferases (e.g. Renilla luciferase, Gaussia luciferase, Oplophorus luciferase, and Cypridina luciferase etc). CLZ analogs may be selected from CLZ-e, CLZ-hcp, CLZ-h, CLZ-f, CLZ-v, CLZ-ip, CLZ-cp, CLZ-n, CLZ-fcp and CLZ-i.

The live transfected cells are incubated with the CLZ cofactor, which can either be in native form or in a chemically modified form (e.g. CLZ-f). CLZ readily traverses the plasma membranes and enters the cell in order to complex with the apophotoprotein. In case of GPCRs, cells containing the reconstituted photoprotein are then exposed to a ligand (e.g, ATP, carbachol, pharmaceutical drugs etc.) for the GPCR, and luminescence is quantified with a luminometer.

The invention also relates to a reconstituted mutant aequorin complex, which comprises the apoaequorin mutant protein, a native or modified synthetic CLZcofactor, and molecular oxygen. Such a reconstituted mutant aequorin complex may be obtained, by known methods in the art, in vitro, in cellulo or in vivo by making the CLZ cofactor available for the mutant apoaequorin (Bioluminescence: Methods and Protocols (Methods in Molecular Biology) 2nd ed. 2009.).

The following examples merely intend to illustrate the present invention.

### EXAMPLES

### Example 1

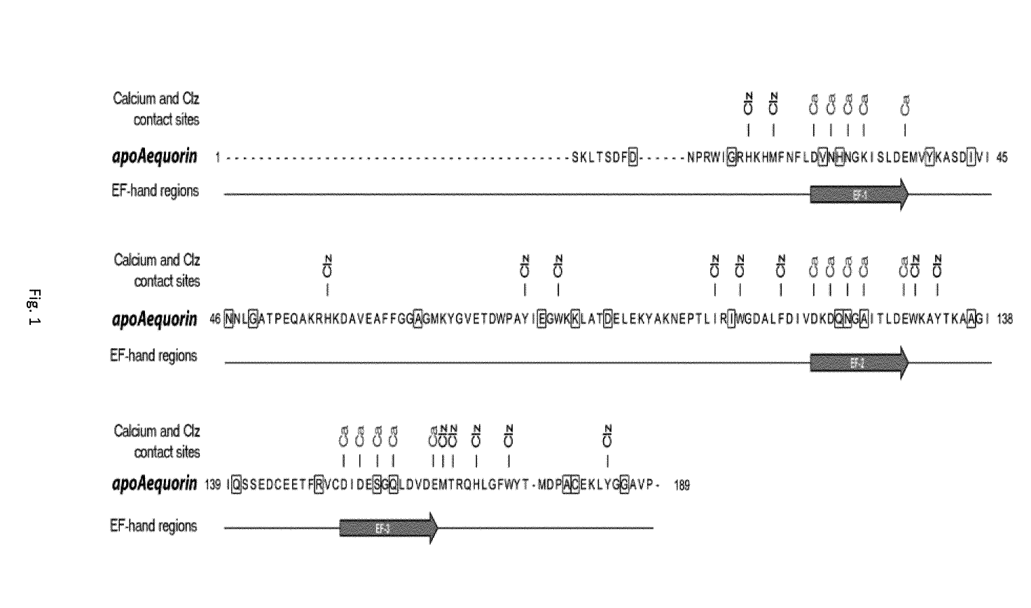

Screening of putative mutated sites for enhanced calcium sensitivity of apoaequorin (Aequorea victoria; PDB code: 1EJ3) was performed, amino acid structure was highlighted as shown in FIG. 1. The apoaequorin sequence is 198 amino acids long. The amino acids in squares represent the summary of mutations performed on aequorin protein sequence in order to alter its calcium affinity. CLZ and calcium amino acids contact sites are shown as Clz and Ca, respectively. Calcium-binding EF-hands are displayed as secondary structure in arrows (EF-1, EF-2, EF-3 regions).

### Example 2

Material and Methods

Site-Directed Mutagenesis of Apoaequorin and Redquorin

The construct of wt-apoaequorin (PDB ID: 1EJ3; SEQ ID NO:1), and the fusion proteins wt-redquorin (wt-Redq) and citrine-aequorin (CitA), subcloned in pTriEx vector, were generated and obtained as generous gift from Prof. Juan Llopis (Bakayan et al. 2011; Bakayan et al. 2015). The fusion construct of GFP-aequorin (GA) was generated in the lab of Prof. Philippe Brûlet (Baubet et al. 2000). The single and double point mutations identified were directly applied on wt-apoaequorin or redquorin constructs using multiple-site directed mutagenesis approach (according to the manufacturer protocol, Agilent technologies). The DNA sequence of all mutants was verified by Sanger sequencing.

Cell Culture and Transfection

HEK-293 cells (kind gift from Dr. Helene Faure) were cultured in Dulbecco's Modified Eagle's Medium (Lonza) supplemented with 2 mM L-glutamine, 10% heat-inactivated fetal bovine serum (FBS) and 100 U/ml of penicillin/streptomycin (Lonza). Cells were routinely maintained at humidified atmosphere of 37° C. and 5% CO₂.

For transfection with foreign DNA vector, cells were seeded at a density of 6×10⁵/cm², and transfected the day after with Lipofectamine 2000 (Invitrogen) according to the manufacturer's recommendations. Before each experiment, cells were washed twice with Hank's Balanced Salt Solution (HBSS) and apoaequorin expressed in different cell lines, was charged with 3 μM CLZ-f or CLZ-native (Biotium) resuspended in OptiMEM I medium (Gibco) and supplemented with 1% FBS for 2-3 hours at 37° C. and 5% CO₂.

Protein Purification from HEK Cells

One day post-transfection, HEK-293 cells (2-3×10⁶ cells) transiently expressing the apoaequorin photoproteins were rinsed (twice) with phosphate-buffered saline (PBS) and collected using a cell scraper, under cold conditions. Cells were harvested at 500×g for 5 minutes and then resuspended and washed once in cold PBS buffer. Subsequently, cells were lysed using a hypo-osmotic buffer composed of 20 mM Tris-HCl (pH 7.5), 10 mM EGTA, and 5 mM ß-mercaptoethanol, prepared in Milli-Q/H₂O and supplemented with a protease inhibitor cocktail (complete-Mini, EDTA-Free, Roche). The cell membranes were broken by two freeze-thaw cycles, followed by few passages through a 25-gauge needle. The resulting lysates samples were then centrifuged at 13,000×g for 20 minutes to remove cell debris and unbroken cells. The 500 μl supernatant containing the photoprotein was recovered and passed through molecular weight cut-offs (AmiconUitra MWCO, 10K and 50K, from Millipore). Columns of 10 kDa were used for apoaequorin mutant proteins and 50 KDa for redquorin mutant proteins. This step allowed for higher purity and buffer exchange by eliminating unwanted proteins, salts and different compounds in the eluted protein samples. The samples were concentrated from, approximately, 500 μl to 20 μl volumes for buffer exchange. The 20 μl concentrated samples were resuspended in 500 μl volume of the desired buffer and this step was repeated twice to ensure high fidelity wash and buffer exchange. The concentrated samples were stored at 4° C. for reconstitution in future assays.

Protein Expression and Purification from E. coli

Some experiments of affinity, emission lifetime and thermostability were repeated on highly purified proteins and gave similar results (Redq/Q159D; Redq/Q159D+A179T, Redq/Q159T, Redq/Q159T+A179T and Redq/Q159T+A123D). Consult more detailed protocol in the following references (Bakayan et al. 2011; Bakayan et al. 2015). Briefly, expression of His-tagged photoproteins was carried out in E. coli using pTriEx-4 plasmid system. Bacterial cells expressing the photoproteins were then lysed and photoproteins were column purified using Ni-NTA HisBind resin (Novagen). An additional purification step was performed using molecular weight cut-offs (AmiconUitra MWCO, 50K, from Millipore) as described in the previous section of cell culture. Protein quantification of the protein samples was performed on Nanodrop machine, by using a combination of absorbance either at 280 nm or at wavelength that corresponds to the maximum extinction coefficient of the fluorescent protein tdTomato in Redq (554 nm). Pure samples were stored at 4° C. for reconstitution in future assays.

Apoaequorin Reconstitution

For in vitro reconstitution, the purified and concentrated samples of apoaequorin and redquorin mutant proteins were buffer-exchanged to 50 mM Tris-HCl (pH 7.5), 150 mM NaCl, 10 mM EGTA, 5 mM ß-mercaptoethanol, supplemented with 5 μM CLZ-f or CLZ-native and incubated overnight at 4° C., in the dark. The next day, samples were passed through molecular weight cut-offs (AmiconUitra MWCO, 10K and 50K, from Millipore), then resuspended in Zero-Ca²⁺ buffer (30 mM MOPS, 100 mM KCl and 10 mM EGTA, pH 7.2) for calcium affinity, emission decay kinetics and spectral analysis assays. As for in cellulo reconstitution, HEK cells transiently expressing the proteins of apoaequorin and aporedquorin mutants were washed twice with Hank's Balanced Salt Solution (HBSS) and incubated in the dark, with 4 μM of the desired CLZ diluted in OptiMEM I medium (Gibco), for 2-3h at 37° C. and 5% CO₂. Cells were washed once and maintained in HBSS at 37° C. for 20 min before the experiment.

Functional Analysis of Aequorin Mutants and Fusions Ca²⁺ Sensitivity Curves.

Purified and reconstituted samples with CLZ-f or CLZ-native already buffered-exchanged to zero-calcium buffer ensured to minimize calcium contamination that could interfere with the measurements. Fifteen microliter aliquot of photoprotein samples (concentration range, 50 to 90 ng/μl) was placed in a home-made luminometer and mixed with 300 μl solution containing the desired concentration of free-Ca²⁺. The solutions of EGTA-buffered with varying free-Ca²⁺ prepared according to the manufacturer instruction for the kit (Molecular Probes, Invitrogen). Integration of the luminescence emission (per second) was measured for 20 seconds (background) before injecting a solution with known [free-Ca²⁺] and lasted until stable or decaying signal (at final-phase) was obtained. At this point, 400 μl of a saturating calcium solution (30 mM MOPS, 100 mM CaCl₂, pH 7.2) was rapidly injected (less than 30 ms) and light integration continued until all aequorin had been consumed and light intensity had gone back to background signal. Luminescence intensity (L) at different [Ca²⁺] was taken as the response peak value in case of decaying signal or as the stable value in case of stable emission. Lmax, was measured by integrating all remaining luminescence signal from that moment (L value) to the end of the experiment. The EC50 values were extracted from a sigmoidal dose-response fit (variable slope) using GraphPad Prism software.

Emission Half-Life.

For assessing the decay kinetics of the mutant proteins of aequorin and redquorin, as well as citrine-aequorin (CitA) and GFP-aequorin (GA), the emission half-life (t_{1/2}) was calculated from the curve with monoexponential decay fit. The recording of the luminescence signal (sampling interval of 30 ms) started before the fast injection of 200 μl of saturating calcium solution (50 mM Tris-HCl, 100 mM CaCl₂, pH 7.5) into fifteen microliter aliquot of protein samples. This resulted in a peak response with complete aequorin consumption and emission decay in less than 8 seconds for all samples.

Spectral Measurement and Analysis.

Fifteen microliter aliquot of photoprotein samples was brought in contact with 200 μl saturating calcium solution (50 mM Tris-HCl, 100 mM CaCl₂, pH 7.5) in a PCR tube to induce light emission. Emitted photons were collected via an optic fiber, guided into a spectrometer (Specim), and captured by an EM-CCD camera (Andor DU-897 back illuminated). Spectral calibration was performed using laser pointers (405 and 650 nm). This home-made setup, from the lab of Prof. Philippe Brûlet, allowed immediate and synchronous spectral analysis (no scanning) of emitted luminescence with 1-nm resolution (Curie et al. 2007).

Results

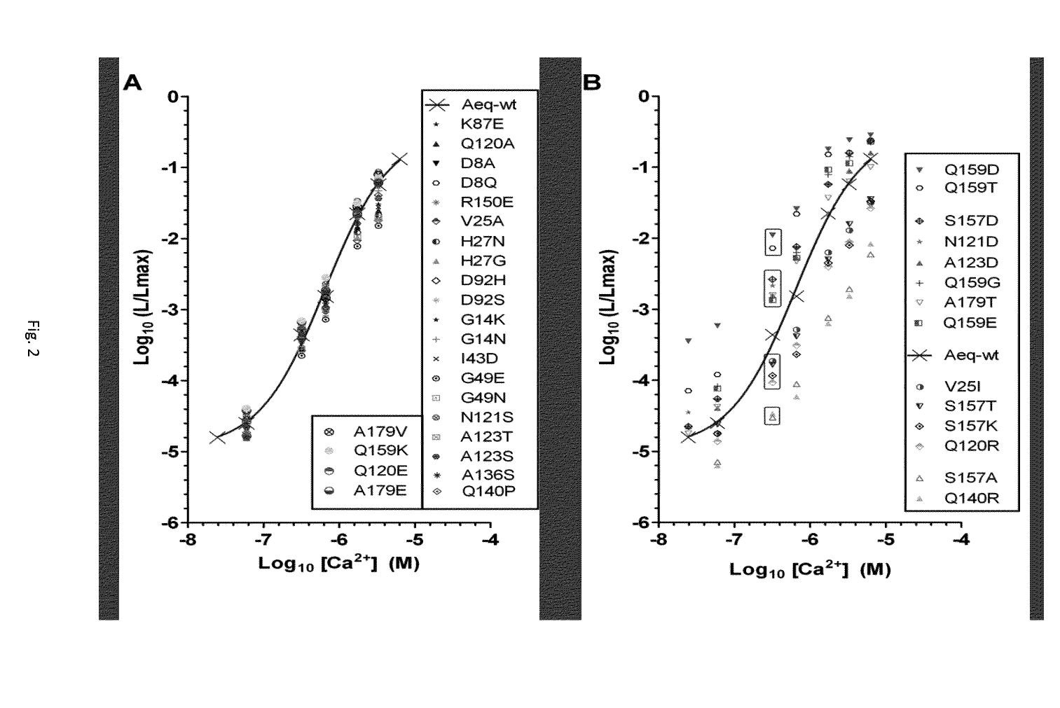

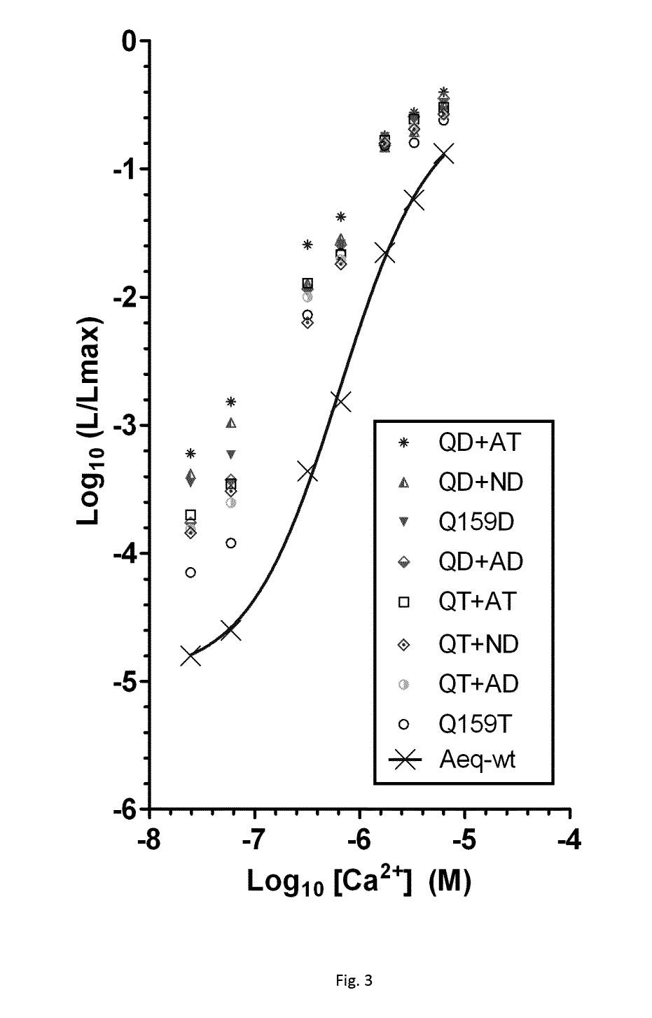

Results of calcium affinity curves of aequorin mutant proteins with single and double mutations are respectively shown on FIG. 2 and FIG. 3. The relationship between calcium concentration ([Ca²⁺]) and fractional bioluminescence intensity (L/Lmax) is displayed in Log values on a linear scale. L and Lmax stand for the peak luminescence intensity at a given [Ca²⁺] and the total luminescence intensity at saturating [Ca²⁺] for the same sample, respectively. Aequorin reconstitution was with CLZ-f.

Analysis with calcium titration curve of aequorin mutants, using increasing calcium doses, resulted in the identification of five groups with different performances. A large group where aequorin affinity to calcium was not significantly altered (FIG. 2A), and four groups with significant change in affinity (FIG. 2B), compared to wt-aequorin (wt-aeq). Surprisingly, two of these groups had specific mutations that resulted in significantly medium-high to high affinity for calcium (e.g. A123D; A179T; Q159D and Q159T,). Whereas, the same position changed to a different amino acid family resulted in unpredicted change of affinity (cf., Q159K vs. Q159D; S157D vs. 5157A; and A179T vs. A179E, FIG. 2B). In fact, some amino acids mutations resulted even in complete opposite and low calcium affinity (e.g. S157A and Q140R), which highlighted the importance and specificity of these point mutations in aequorin luminescent reaction. For Aeq-wt, a sigmoidal curve fit is traced for comparison. R square of the fit goodness was higher than 0.994.

Detailed analysis of the affinity curves of these groups showed a significant decrease in calcium EC50, where the mutations Q159T (310 nM) and Q159D (330 nM) have shown the highest affinity, followed by S159D (322 nM), A179T (330 nM) and A123D (374 nM), Table 3. Calculating the relative intensity at pCa 6.5 and pCa 7.2 of the these single mutants revealed that Q159D and Q159T had the highest luminescence output at these calcium doses, being 25 and 16 time higher than wt-aeq. The mutant Q159D had drastically high luminescence at minute calcium dose of pCa 7.2, with a recorded intensity up to 23 times higher than that of wt-aeq, at equivalent concentration, Table 3, which reflects its potential utility and sensitivity at detecting small calcium concentrations. Based on random mutagenesis, one previous study has reported few mutations, E35G and V44A, to increase calcium affinity in aequorin. However, such mutants were accompanied also with very long decay kinetics, emission half-life (t½) of 40 and 34 s, respectively, compared to 800 ms in wt-aeq (Tsuzuki et al. 2005). This slow decay property hampered from exploiting these mutations for calcium imaging applications in cells. Therefore, aequorin mutants identified herein, with increased affinity (Table 3), were assessed for the emission decay half-life at saturating calcium conditions. Surprisingly, the half-lives of Q159D and A179T mutations were significantly faster than wt-aeq, (779 ms vs. 906 ms). The rest of mutations resulted in relatively lower decay rate except for aequorin mutant N121D that gave slightly higher decay half-life (957 ms vs. 906 ms).

TABLE 3
Summarv of the properties of single mutation aeauorin mutant proteins with increased calcium sensitivity.

| | | Calcium | | | Decay | Spectral |
|---|---|---|---|---|---|---|
| | | sensitivity, | Relative intensity^{a} | | kinetics, | emission peak |
| | Mutation | EC50 (nM)^{a} | at pCa 6.5 | at pCa 7.2 | t_{1/2} (ms)^{b} | (nm) |
| | Aeq-wt | 659 ± 23 | 1 | 1 | 906 ± 53 | 476 |
| Single mutants with | Q159D | 330 ± 8 | 25.1 | 23.4 | 794 ± 42 | 478 |
| higher affinity | Q159T | 249 ± 13 | 16.6 | 4.8 | 866 ± 23 | 477 |
| | S157D | 322 ± 20 | 6.0 | 2.2 | 835 ± 47 | 475 |
| | N121D | 400 ± 14 | 4.9 | 2.1 | 957 ± 30 | 477 |
| | A123D | 374 ± 9 | 3.6 | 1.6 | 862 ± 48 | 476 |
| | Q159G | 492 ± 10 | 3.3 | 3.2 | 830 ± 37 | 478 |
| | A179T | 330 ± 19 | 3.0 | 1.3 | 779 ± 33 | 477 |

Results from Table 3 were obtained with aeauorin mutant proteins reconstituted with CLZ-f.
Calcium sensitivity (represented in EC50) was calculated from sigmoidal curve fit, n was at least 3. Values are displayed as mean ± SD.
Luminescence relative intensity (compared to Aeq-wt) was deduced from data points of L/Lmax for two calcium concentrations, pCa 6.5 and pCa 7.2.
^{a}the R square of the goodness of the curve fit was higher than 0.994.
The decay kinetics was assayed by fast- flow injection of a saturating calcium solution and recording of the luminescence signal decay at a sampling interval of 30 ms. Mean values ± SD were calculated from a monoexponential decay curve fit.
^{b}the R square of the goodness of the decay curve fit was higher than 0.999.

Further, the combination of two potential single mutations resulted in aequorin mutants with additional enhanced properties. Indeed, data from calcium titration curves revealed that all three double mutations (N121D, A123D and A179T) applied on aequorin mutants (Q159D and Q159T) resulted in further increase of calcium affinity (cf. Q159D, Q159T vs. Q159D+A179T and Q159T+A123D), FIG. 3 and Table 4. In fact, the aequorin double mutations Q159D+A179T and Q159T+A123D showed the lowest calcium EC50 of each category, 216 nM and 279 nM, respectively. In addition, the decay half-lives of all double mutants were significantly improved compared to wt-aeq, especially Q159D+A179D and Q159D+A123D, being the fastest of all, 612 ms and 620 ms, respectively (Table 4). Analysis of spectral emission, represented by emission peaks, of all the aequorin mutants did not highlight any significant change relative to wt-aeq. This may suggest that these mutations affected in a specific manner the reaction dynamics of calcium/aequorin and not the stability of CLZ moiety responsible for light emission.

TABLE 4
Summary of the properties of double mutations aequorin mutant proteins with increased calcium sensitivity.

| | | | | | | Spectral |
|---|---|---|---|---|---|---|
| | | Calcium | | | Decay | emission |
| | | sensitivity, | Relative intensity^{a} | | kinetics, | peak |
| | Mutation | EC50 (nM)^{a} | at pCa 6.5 | at pCa 7.2 | t_{1/2} (ms)^{b} | (nm) |
| | Aeq-wt | 659 ± 23 | 1 | 1 | 906 ± 53 | 476 |
| Double mutants with | QD + AT | 216 ± 26 | 58.5 | 60.8 | 750 ± 50 | 478 |
| higher affinity | QD + ND | 295 ± 17 | 29.0 | 41.7 | 738 ± 28 | 477 |
| | QD + AD | 271 ± 15 | 26.2 | 15.0 | 612 ± 26 | 475 |
| | QT + AT | 281 ± 25 | 29.0 | 14.0 | 650 ± 68 | 478 |
| | QT + ND | 337 ± 10 | 14.4 | 12.2 | 680 ± 55 | 478 |
| | QT + AD | 279 ± 16 | 22.8 | 10.0 | 620 ± 33 | 476 |

The following abbreviations are used for clarity: OD + AT (Q159D + A179T), QD + ND (Q159D + N121D), QD + AD (Q159D + A123D), QT+AT (Q159T + A179T), QT + ND (Q159T + N121D), QT + AD (Q159T + A123D).
Results from Table 4 were obtained with aequorin mutant proteins reconstituted with CLZ-f.
Calcium sensitivity (represented in EC50) was calculated from sigmoidal curve fit, n was at least 3. Values are displayed as mean ± SD.
Luminescence relative intensity (compared to Aeq-wt) was deduced from data points of L/Lmax for two calcium concentrations, pCa 6.5 and pCa 7.2.
^{a}the R square of the goodness of the curve fit was higher than 0.994.
The decay kinetics was assayed by fast-flow injection of a saturating calcium solution and recording of the luminescence signal decay at a sampling interval of 30 ms. Mean values ± SD were calculated from a monoexponential decay curve fit.
^{b}the R square of the goodness of the decay curve fit was higher than 0.999.

### Example 3

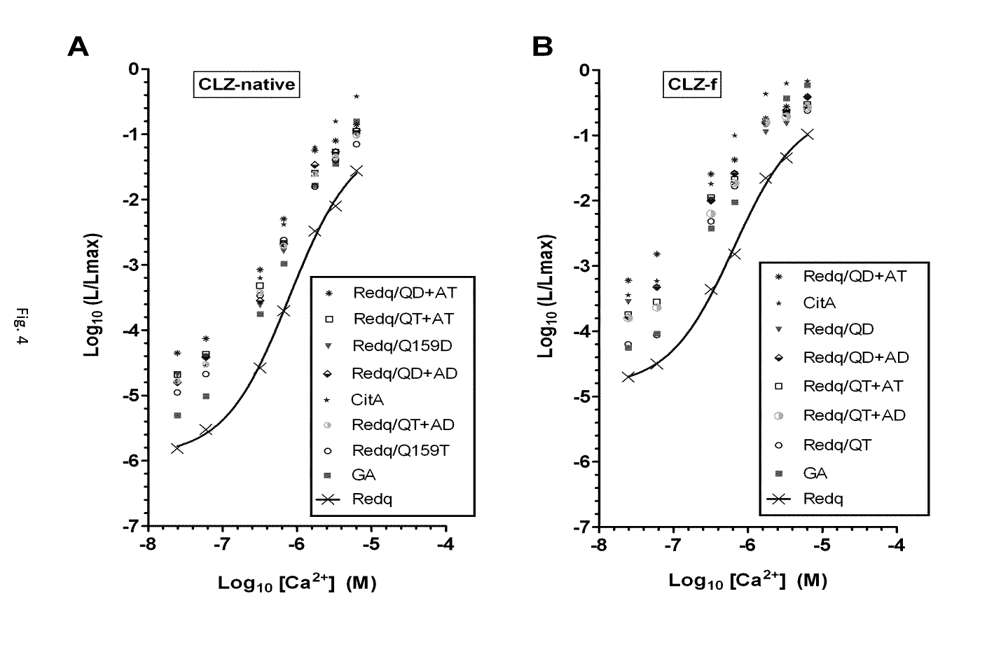

The same material and methods as example 2 was used herein. In this example we tested the performance of the described aequorin mutations on redquorin in order to boost up its sensitivity for calcium and thus its bioluminescence output. Six mutants of redquorin have been created and assessed for their calcium sensitivity, relative intensity and emission decay either with CLZ-native or CLZ-f. As a result, all redquorin mutants have shown significant increase in calcium affinity, independently of the CLZ cofactor used (FIG. 4). The calibration curve of the mutant Redq/QD+AT was the closest to CitA, whereas Redq/QT was the most similar to GA.

Results of properties of redquorin mutant proteins, redquorin-wt (Redq), Citrine-aequorin fusion (CitA) and GFP-aequorin fusion (GA) (FIG. 4) are detailed in Table 5 below. All the aequorin photoproteins, Redq, Redq mutants, GA and CitA were reconstituted either with CLZ-native (FIG. 4.A; top section of table 5) or CLZ-f (FIG. 4.B; bottom section of table 5). For more details on calcium sensitivity (represented in EC50), luminescence relative intensity and decay kinetics, refer to table 3. ^{a} is the R square of the goodness of the curve fit was higher than 0.995. n was at least 3. ^{b} is the R square of the goodness of the decay curve fit was higher than 0.999. n was at least 3.

In terms of relative intensity to redquorin (at pCa 6.5), Redq/QD+AT proved to be with the highest light output, between 30 and 60 times more intensity, depending on the type of CLZ cofactor, Table 5. Comparably, other double mutations (Redq/QT+AT and Redq/QT+AD) showed significant increase in relative intensity, between 14 and 25 times compared to redquorin. Interestingly, the redquorin mutants holding the mutation Q159T (Redq/QT; Redq/QT+AT and Redq/QT+AD), generally did not demonstrate a noteworthy difference in relative intensity when changing the CLZ cofactor (cf. values 13 vs. 11; 18.3 vs. 25.6 and 14.4 vs. 14.4; Table 5). Similarly, the relative intensity values for the fusion GA did not change considerably when testing the two CLZ cofactors, in contrast to CitA. Regarding emission half-life, most double mutants resulted in relatively similar to faster emission kinetics compared to redquorin. With CLZ-native, the redquorin mutants Redq/QT+AD and Redq/QT are the fastest, having half-lives of 670 ms and 705 ms, respectively. While with CLZ-f, the mutants Redq/QD+AT and Redq/QT+AD showed the smallest half-live values of 913 ms and 950 ms, respectively, Table 5. In fact, these particular mutations have improved redquorin properties in terms of affinity and emission kinetics, making it interestingly similar to GA and CitA. Such findings would allow the use of these redquorin mutants in applications of dual color calcium detection, deep tissue imaging, as well as in assays that require the use of CLZ-native while keeping significant detection sensitivity.

TABLE 5
Summary of the propeties of redquorin mutant proteins with increased calcium sensitivity.

| | | | | | Decay | Spectral |
|---|---|---|---|---|---|---|
| | | Calcium sensitivity, | Relative intensity^{a} | | kinetics, t_{1/2} | emission |
| | Mutation | EC50 (nM)^{a} | at pCa 6.5 | at pCa 7.2 | (ms)^{b} | peak (nm) |
| CLZ-native | Redq | 859 ± 45 | 1.0 | 1.0 | 1 203 ± 70 | 582 |
| | Redq/Q159D | 680 ± 33 | 9.1 | 13.2 | 980 ± 66 | 582 |
| | Redq/QD + AT | 515 ± 20 | 32.1 | 24.5 | 913 ± 39 | 582 |
| | Redq/QD + AD | 621 ± 44 | 10.8 | 12.8 | 1 120 ± 46 | 582 |
| | Redq/Q159T | 478 ± 39 | 13.0 | 7.1 | 1 103 ± 50 | 582 |
| | Redq/QT + AT | 605 ± 16 | 18.3 | 14.1 | 990 ± 87 | 582 |
| | Redq/QT + AD | 577 ± 33 | 14.4 | 10.0 | 950 ± 56 | 582 |
| | CitA | 573 ± 45 | 24 | 9.5 | 794 ± 46 | 529 |
| | GA | 630 ± 29 | 6.8 | 3.2 | 852 ± 64 | 509 |
| CLZ-f | Redq | 659 ± 23 | 1.0 | 1.0 | 880 ± 68 | 582 |
| | Redq/Q159D | 290 ± 18 | 23.9 | 14.9 | 770 ± 88 | 582 |
| | Redq/QD + AT | 252 ± 40 | 58.5 | 48.3 | 740 ± 55 | 582 |
| | Redq/QD + AD | 300 ± 26 | 22.8 | 15.0 | 750 ± 66 | 582 |
| | Redq/Q159T | 296 ± 13 | 11.0 | 2.8 | 705 ± 36 | 582 |
| | Redq/QT + AT | 284 ± 30 | 25.6 | 9.0 | 810 ± 61 | 582 |
| | Redq/QT + AD | 336 ± 11 | 14.4 | 7.4 | 670 ± 50 | 582 |
| | CitA | 266 ± 18 | 41.1 | 18.7 | 680 ± 72 | 529 |
| | GA | 463 ± 46 | 8.6 | 2.9 | 700 ± 48 | 509 |

The following abbreviations are used for clarity: QD + AT(Q159D + A179T), QD + AD (Q159D + A123D), QT + AT (Q159T + A179T), QT + AD (Q159T + A123D).
For more details on calcium sensitivity (represented in EC50), luminescence relative intensity and decay kinetics, refer to table 3.
^{a}the R square of the goodness of the curve fit was higher than 0.995. n was at least 3.
^{b}the R square of the goodness of the decay curve fit was higher than 0.999. n was at least 3.

### Example 4

Thermostability

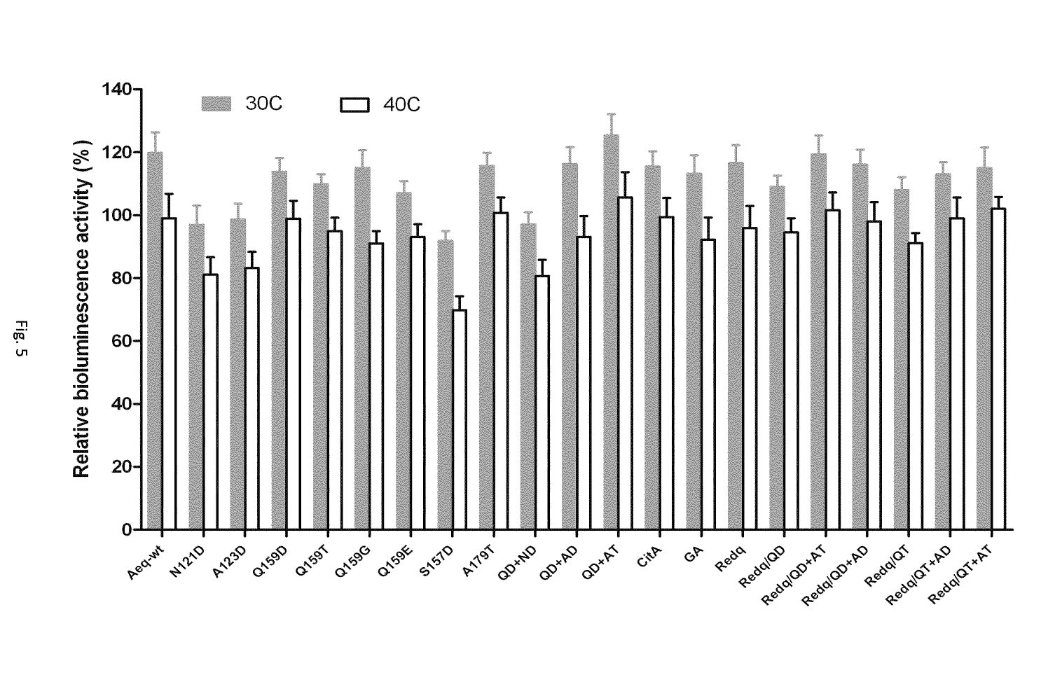

Thermostability of aequorin and redquorin mutant with increased affinity for calcium were compared to wt-aequorin (FIG. 5). The purified photoprotein samples were reconstituted with CLZ-f (as detailed earlier) and let to calibrate at room temperature (20-24° C.) for 20 min before taken the first measurement of total counts using saturating calcium solution (50 mM Tris-HCl, 100 mM CaCl₂, pH 7.5). Samples were then incubated at two different temperatures (30 to 40° C.) for 30 min, and let equilibrate at room temperature for 15 minbefore taken the second measurement of total counts in the sample. The relative bioluminescence activity at each temperature was calculated by the ratio 2^{nd} counts (at target T° C.)/1^{st} counts (at initial T° C.) multiplied by 100.

Table 6 highlights the analysis of thermostability data obtained at two temperatures for aequorin and redquorin mutants, as well as CitA and GA. The values represent the degree of change in luminescence activity compared to wt-aeq, at each temperature. All values are relative to wt-aequorin (at each temperature) and were calculated by dividing the obtained data for each mutant/variant by the data for wt-aeq (as reference). The data were taken from FIG. 5.

TABLE 6
Thermostability data obtained at two temperatures for aequorin and redquorin
mutants, as well as CitA and GA.

| | wt- | | | | | | |
|---|---|---|---|---|---|---|---|
| Temperature | Aeq | N121D | A123D | Q159D | Q159T | Q159G | Q159E |
| 30° C. | 1.00 | 0.81 | 0.82 | 0.95 | 0.92 | 0.96 | 0.89 |
| 40° C. | 1.00 | 0.82 | 0.84 | 1.00 | 0.96 | 0.92 | 0.94 |

| | S157D | A179T | QD + ND | QD + AD | QD + AT | CitA | GA |
|---|---|---|---|---|---|---|---|
| 30° C. | 0.77 | 0.96 | 0.81 | 0.97 | 1.05 | 0.96 | 0.95 |
| 40° C. | 0.71 | 1.02 | 0.81 | 0.94 | 1.07 | 1.00 | 0.97 |

| | | Redq/ | Redq/QD + | Redq/QD + | | Redq/QT + | Redq/QT + |
|---|---|---|---|---|---|---|---|
| | Redq | QD | AT | AD | Redq/QT | AD | AT |
| 30° C. | 0.97 | 0.91 | 1.00 | 0.97 | 0.90 | 0.94 | 0.96 |
| 40° C. | 0.97 | 0.95 | 1.03 | 0.99 | 0.92 | 1.00 | 1.03 |

The following abbreviations are used for clarity: QD + AT(Q159D + A179T), QD + ND (Q159D + N121D), QD + AD (Q159D + A123D), QT + AT (Q159T + A179T), QT + AD (Q159T + A123D).

The point mutations of aequorin and redquorin reported herein have certainly affected the structure and dynamics of aequorin and thus may also affect its activity at elevated temperatures. Therefore, in an experimental procedure, the luminescence activity of aequorin mutants and its derivatives was registered after incubation at two representative temperatures (FIG. 5). Consequently, the point mutation S157D resulted in the lowest thermostability, up to 29% less activity compared to wt-aeq, followed by N121D and A123D mutations (19% less activity), Table 6. As for the double mutations, the mutant QD+ND retained the lowest activity (20% less) compared to wt-aeq. Generally, most of the mutants maintained similar to slightly less activity at the tested temperatures relative to wt-aequorin.

All these findings together add to the potential suitability of these aequorin and redquorin mutant variants for detecting calcium in live cells with higher sensitivity and fidelity.

### Example 5

Redquorin mutants based cellular assay for activation of endogenous P2Y2 receptor in CHO cells

Materials and Methods

Cell Culture and Transfection

CHO cells were grown in Dulbecco's modified Eagle's medium (DMEM)/F12 medium supplemented with 10% fetal calf serum at 37° C., 5% CO₂ for 24 h.

For transfection with foreign DNA vector, cells were seeded at a density of 6×10⁵/cm², and transfected the day after with Lipofectamine 2000 (Invitrogen) according to the manufacturer's recommendations. Before each experiment, cells were washed twice with Hank's Balanced Salt Solution (HBSS) and apoaequorin expressed in different cell lines, was charged with 3 μM CLZ-f or CLZ-native (Biotium) resuspended in OptiMEM I medium (Gibco) and supplemented with 1% FBS for 2-3 hours at 37° C. and 5% CO₂.

Stably prepared CHO cells were loaded with 5 μM coelenterazine in 130 mM NaCl, 5 mM KCl, 1 mM MgCl₂, 2 mM CaCl₂, 5 mM NaHCO₃, 20 mM N-(2-hydroxyethyl) piperazine-N′-ethanesulfonic acid (HEPES), pH 7.4 for 4 h at ambient temperature.

CHO Cells Stably Expressing Photoproteins

To produce stable CHO cell lines, the cells transfected with GA and Redquorin variant plasmids were seeded on six-well plates and grown in the same medium with addition of Geneticin sulfate (G418) (Gibco, UK) at 1 mg/ml under the same conditions with replacement of the medium by fresh medium every 2 days. After 7-10 days of selection, CHO cells were transferred into a medium without antibiotic. To select clones with the highest bioluminescence activity, one run of limiting dilution was performed. CHO cells after G418 selection were seeded on 96-well plates (approximately 0.5 cells per well) and grown in DMEM/F12 medium supplemented with 10% fetal calf serum at 37° C., 5% CO₂ for up to 80-90% confluence. Before the bioluminescence measurements, the plates with monoclones of CHO cells expressing the corresponding photoprotein were duplicated and grown for up to 90-100% confluence. Then, the medium was removed and cells were loaded with coelenterazine, and bioluminescence in each well was measured according to the procedure described earlier. As a result, the CHO cell clones with the highest bioluminescence activity were selected.

Assay of Activation of Endogenous P2Y2 Receptor in CHO Cells

The CHO cell lines stably expressing each photoprotein were used in assay of activation of endogenous P2Y2 receptor by ATP. The day before the measurements, the corresponding cells were seeded on 96-well plates with DMEM/F12 medium supplemented with 10% fetal calf serum and were grown at 37° C. with 5% CO₂ for up to 90-100% confluence. Then, the medium was replaced by 100 μl of coelenterazine solution per well (5 μM coelenterazine in 130 mM NaCl, 5 mM KCl, 1 mM MgCl₂, 2 mM CaCl₂, 5 mM NaHCO₃, 20 mM HEPES, pH7.4) and the cells were incubated at 23° C. for 4 h. Emission from photoproteins in the cells was measured using a Mithras LB 940 plate luminometer. The ATP was injected at a given concentration to trigger an intracellular Ca²⁺ response in the cells and the measurement started immediately. The bioluminescence was integrated over a time interval of 0.05 s for 30 s.

Results

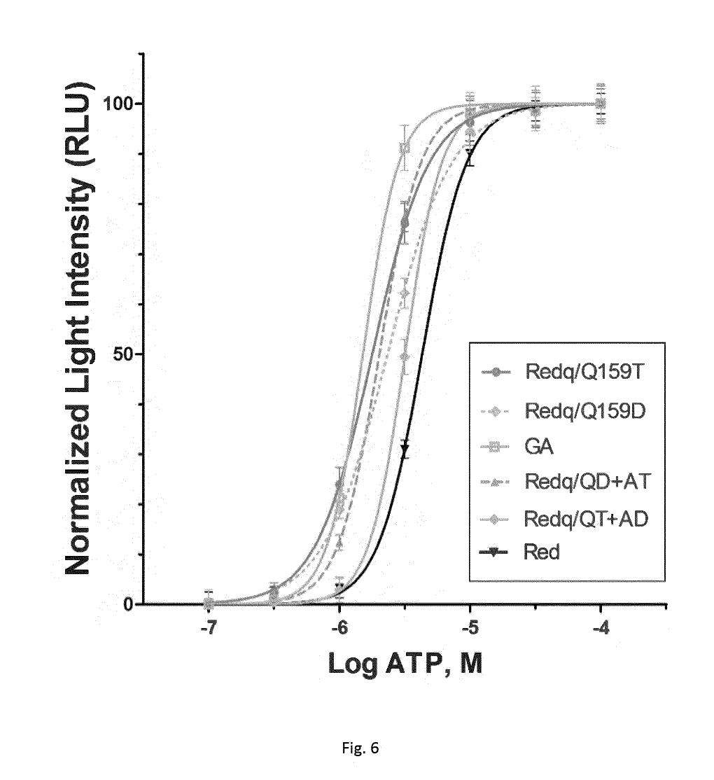

Ca²⁺-regulated photoproteins are mainly applied to detect intracellular Ca²⁺ in mammalian cells. Here, a selection of red Ca²⁺-sensor mutants were tested for their ability to be expressed and converted into active photoproteins in CHO cells, and in the assay of activation of endogenous cellular receptors. All red sensor variants showed similar cytoplasmic and homogeneous expression pattern. CHO cells stably expressing the corresponding red sensor mutant were incubated with coelenterazine native and after cell lysis with 1% Triton X-100, bioluminescence was measured by integrating the light signal for 10s. The red sensor mutants revealed significant and similar total bioluminescence activities with comparable efficiency for the conversion of apophotoprotein into active Ca²⁺-sensitive sensor. The assay for the activation of endogenous P2Y2 purinergic receptor was performed with the same CHO cells stably expressing GA, Redq, Redq/Q159T, Redq/Q159D, Redq/QD+AT and Redq/QT+AD. In CHO cells, the activation of P2Y2 receptors by external application of ATP doses leads to activation of the IP3 (inositol 1, 4, 5-triphopsphate) signaling pathway and an increase in the cytoplasmic Ca²⁺ concentration. Upon P2Y2 receptor activation by ATP, CHO cells stably expressing aequorin-based Ca²⁺ sensors showed a prominent dose-dependent response (FIG. 6). Detailed analysis of the ATP response is presented in Table 7.

TABLE 7
Performance properties of redquorin mutants in
cellular assay for P2Y2 receptor activation

| Ca²⁺ sensor variant | [ATP] DL (nM) | EC₅₀ (μM) | Z-factor |
|---|---|---|---|
| Redq/Q159T | 398 ± 17 | 1.7 ± 0.2 | 0.76 |
| Redq/Q159D | 417 ± 16 | 2.3 ± 0.2 | 0.68 |
| Redq/QD + AT | 562 ± 20 | 2 ± 0.2 | 0.67 |
| Redq/QT + AD | 1230 ± 51 | 3.1 ± 0.4 | 0.82 |
| Redq | 1585 ± 53 | 4.3 ± 0.3 | 0.56 |
| GA | 603 ± 25 | 1.5 ± 0.2 | 0.62 |

DL: detection limit;
EC50: Half-maximal effective concentration

The values of half-maximal effective concentrations (EC50) for P2Y2 activation determined with most of CHO cell lines were in close accordance with values from earlier reports [Gealageas et al., 2014, Malikova et al., 2014]. In particular, the red light cell line Redq/Q159T gave closely matching value of 1.7 μM compared with the commonly used green light cell line GA (1.5 μM). However, for the cell lines with native Redq and Redq/QT+AD mutant, EC50 values were around double, mainly due to lower ATP detection limit capacity (Table 7). The most sensitive of all, for ATP presence, was the CHO cell line Redq/Q159T, by detecting down to 398 nM [ATP].

In high-throughput screening assays, a high number of compounds are evaluated, and thus the assays need to be robust and reproducible over time. Hence, the needs for at least one strict assay validation parameter that assures high quality data and suitability of the system. For validating this Ca2+ sensor-based assay, ATP experiments were performed at the maximum and minimum response levels in order to ensure that the signal window is adequate to detect effective active ATP. Accordingly, the Z-factor was calculated for each pair of CHO cell line/ATP response, which reflects the assay signal dynamic range and the data variation associated with the signal measurements. Values of ≥0.6 are commonly considered to indicate a valuable assay (which is comparable to a signal window 3). The data showed that the cell line Redq/QT+AD performed best in terms of signal measurements (value of 0.82) although this CHO cell line lacks sensitivity in [ATP] detection limit. The best-second high quality data were from the cell line CHO/Redq/Q159T (value of 0.76), which performs similarly well in detecting ATP in nanomolar range (Table 7). It is therefore concluded that these mutations improved significantly the properties of the wildtype red Ca²⁺-sensor redquorin for its use in cellular assays.

### Example 6

Expression of Redquorin Mutant in Pyramidal Neurons of Acute Brain Slices

Materials and Methods

Preparation and Production of Recombinant Viral Vectors

Recombinant Sindbis viruses were prepared and used to express Redquorin mutants and GA in neurons of brain slices as described (Drobac et al., 2010), (Tricoire and Lambolez, 2014). The coding sequences of Redquorin mutants and GA were first subcloned in the plasmid pSinRep5 (Invitrogen) upstream to the polyA signal. The resulting pSinRep5 plasmids encoding the fusion protein sensors as well as the helper plasmid pDH26S (Invitrogen) were then submitted to in vitro transcription to prepare capped RNA using the Megascript SP6 kit (Ambion). Next, BHK-21 (baby hamster kidney; CCL-10; ATCC) cells were electroporated with both sensors-encoding and helper viral RNAs, and maintained for 24 hr at 37° C., 5% CO₂ in DMEM containing 5% fetal calf serum. Recombinant pseudovirions were harvested by collecting the cell supernatant and were stored at −80° C.

Preparation and Viral Transduction of Neocortical Slices

All experiments were carried out in accordance with the guidelines published in the European Communities Council Directive of 24 Nov. 1986 (86/609/EEC). Parasagittal sections (250 μm-thick) of cerebral cortex were prepared from young C57BL/6J mice (10-14 postnatal days old) as described [Tricoire and Lambolez, 2014]. The slices were incubated at room temperature for 30 min in artificial cerebrospinal fluid (ACSF) containing (in mM): 126 NaCl, 2.5 KCl, 1.25 NaH₂PO₄, 2 CaCl₂, 1 MgCl₂, 26 NaHCO₃, 20 glucose, 5 pyruvate, 1 kynurenic acid and oxygenated with a mixture of 95% O₂/5% CO₂. The slices were then transferred onto a Millicell CM membrane (Millipore) pre-equilibrated with culture medium (50% MEM, 50% HBSS, 6.5 g/litre glucose, 10 U/ml penicillin, 10 μg/ml streptomycin). Transduction of the slices with Sindbis pseudovirions as DNA delivery vehicles was carried out as described (Drobac et al., 2010; Tricoire and Lambolez, 2014). The apophotoproteins in Redquorin mutants and GA were activated overnight by supplementing the culture medium with 10 OA native coelenterazine. Next day, cortical slices were pre-incubated for at least 1 hr in ACSF before being transferred to the imaging chamber where ACSF was continually perfused at a rate of 1-2 ml/min.

Bioluminescence Imaging Procedure

Bioluminescence imaging was performed at room temperature using an intensified CDD video camera (ICCD225; Photek) controlled by the IFS32 software (Photek), and mounted on an upright microscope (BX51WI, Olympus) equipped with water immersion objectives ×10 (NA=0.3) and ×20 (NA=0.95). Prior to bioluminescence imaging, fluorescence images of GA- or Redquorin-transduced slices were acquired in the “bright field” mode of the camera using GFP (Semrock) or Cy3ET (Chroma) filter sets. Bioluminescence imaging was performed in “bining slice” mode at a rate of 25 frames per second in complete darkness and without emission filter to maximize photon capture. Data were collected, stored and visualized with the IFS32 software.

Results

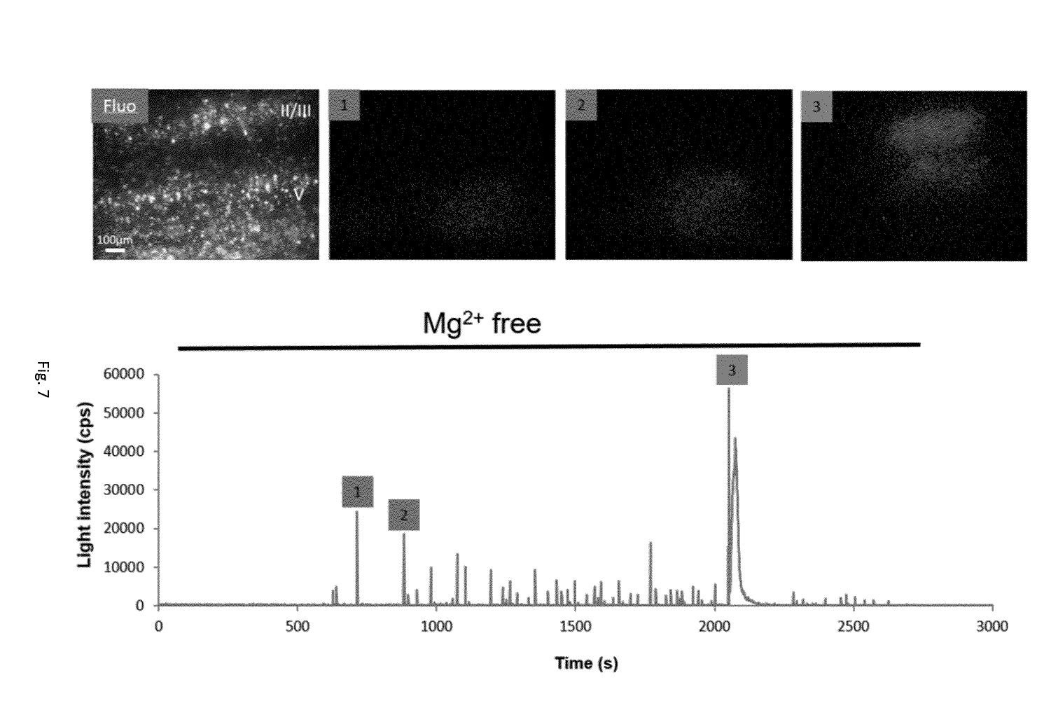

Complying with the 3R principles for animal experimentation, the number of animals scarified for these experiments was restricted. In addition to Redquorin WT and GFP-aequorin (GA), the performance of the new Redquorin mutants was only limited to testing the mutant RedquorinQ159T, As detailed in methods section, the neocortical slices were maintained alive overnight and transduced with the Sindbis viral particles for the expression of Redquorin WT, its mutant RedquorinQ159T and GA. The Sindbis virus is known to be neurotropic with a tropism to infect neuronal cells of the nervous system. Indeed, the expression of the Ca²⁺ sensors visualized by fluorescence showed high efficiency of transducing the neocortex with high and specific expression levels in the pyramidal neurons (FIG. 7, fluorescence image). Compared to previous results obtained by other genetically encoded sensors (Gervasi et al., 2007; Drobac et al., 2010) and GA, the expression of Redquorin WT and its mutant Q159T was homogeneously distributed in the cytoplasm.

Responses of the Ca²⁺ Sensor Mutant to Depolarization in Brain Slices

The transduced acute brain slices expressing the sensors were then perfused with artificial cerebrospinal fluid (ACSF) without Mg²⁺. The response of Redquorin mutant to Ca²⁺ transient was examined by using bioluminescence imaging and was compared to Redquorin WT and GA. Before the threshold for depolarization, the recorded imaging intensity was very weak consistent with the concept of low background luminescence of photoproteins. Soon after depleting Mg²⁺, spontaneous and synchronized light flashes revealing Ca²⁺ activity after depolarization of subsets of neurons were detected and imaged with the Ca²⁺ sensors GA and Redquorin mutant (FIG. 7, time between 500 and 2000 sec). Both sensors, although with different emission color hues, repeatedly gave similar and undistinguishable profiles in terms of signal frequency and amplitude. Interestingly, after minutes of exposure to Mg²⁺-free ACSF, it was frequently observed a slowly self-propagating wave of depolarization across the entire neocortex (FIG. 6, time after 2000 sec). In fact, several clinical and neuroimaging conclusions support such phenomenon known as cortical spreading depression to correlate with neurological symptoms in migraine aura. The mutant RedquorinQ159T showed the ability to image these waves and performed as well as its analogue GA, which is a commonly used Ca²⁺ sensor with green emission spectrum. It is noteworthy that experiments with Redquorin WT failed to reproduce the same response profiles being showing twice less bright intensity. As previously reported in neuronal cultures (Baubet et al., 2000; Rogers et al., 2005; Drobac et al., 2010), RedquorinQ159D allowed imaging of physiological Ca²⁺ signals in neuronal brain slices with significant signal-over-background ratio. Moreover, it has the potential to be a suitable sensor for imaging evoked Ca²⁺ transients at single-cell level.

### BIBLIOGRAPHIC REFERENCES

Ausubel et al. in “Current Protocols in Molecular Biology”, John Wiley a Sons (2011).
Bakayan, A., B. Domingo, et al. (2015). “Imaging Ca(²⁺) activity in mammalian cells and zebrafish with a novel red-emitting aequorin variant.” Pflugers Arch 467(9): 2031-2042.
Bakayan, A., C. F. Vaquero, et al. (2011). “Red fluorescent protein-aequorin fusions as improved bioluminescent Ca²⁺ reporters in single cells and mice.” PLoS One 6(5): e19520.
Baubet V, Le Mouellic H, Campbell A K, Lucas-Meunier E, Fossier P, Brulet P. 2000. Chimeric green fluorescent protein-aequorin as bioluminescent Ca21 reporters at the single-cell level. PNAS USA. 97:7260-7265.
Bovolenta, S., Foti, M., Lohmer, S. and Corazza, S. (2007). Development of a Ca(2+)-activated photoprotein, Photina, and its application to high-throughput screening. J. Biomol. Screen 12, 694-704.).
Curie, T., K. L. Rogers, et al. (2007). “Red-shifted aequorin-based bioluminescent reporters for in vivo imaging of Ca²⁺ signaling.” Mol. Imaging 6(1): 30-42.
David R. O'Reilly et al., Oxford University Press, USA, (1992).
Day r. n.; Davidson m. w., Chemical society reviews, vol. 38, 2009, pages 2887-2921
de la Fuente S, Fonteriz R I, de la Cruz P J, Montero M, Alvarez J., Mitochondrial free [Ca(²⁺)] dynamics measured with a novel low-Ca(²⁺) affinity aequorin probe (2012).
Dikici E., X. Qu, L. Rowe, L. Millner, C. Logue, S. K. Deo, M. Ensor, and S. Daunert, Aequorin variants with improved bioluminescence properties (2009a), Protein Eng Des Sel. 2009 April; 22(4): 243-248.
Dikici E, et Daunert S Nature Chemical Biology 5, 70-71 (2009b)
Drobac, E., Tricoire, L., Chaffotte, A.-F., Guiot, E., a Lambolez, B. (2010). Ca2+ imaging in single neurons from brain slices using bioluminescent reporters. Journal of Neuroscience Research, 88(4), 695-711.
Eremeeva E V, Markova S V, Frank L A, Visser A J, van Berkel W J, Vysotski E S. (2013a) Bioluminescence and spectroscopic properties of His-Trp-Tyr triad mutants of obelin and aequorin. Photochem Photobiol Sci 12: 1016-1024.
Eremeeva E V, Markova S V, van Berkel W J, Vysotski E S. (2013b), Role of key residues of obelin in coelenterazine binding and conversion into 2-hydroperoxy adduct, Journal of Photochemistry and Photobiology B: Biology 127 (2013) 133-139
Gealageas, R., Malikova, N. P., Picaud, S., Borgdorff, A. J., Burakova, L. P., Brûlet, P., Vysotski, E. S., Dodd, R. H., (2014). Bioluminescent properties of obelin and aequorin with novel coelenterazine analogues. Analytical and Bioanalytical Chemistry, 406(11), 2695-2707.
Gervasi N, Hepp R, Tricoire L, Zhang J, Lambolez B, Paupardin-Tritsch D, Vincent P. 2007. Dynamics of protein kinase A signaling at the membrane, in the cytosol, and in the nucleus of neurons in mouse brain slices. J Neurosci 27:2744-2750.
Inouye and Sahara, Protein Express. Purif., 53: 384-389 (2007);
Kendall J M, Dormer R L, Campbell A K (1992) Targeting aequorin to the endoplasmic reticulum of living cells. Biochem Biophys Res Commun 189: 1008-1016
Malikova, N. P., Burakova, L. P., Markova, S. V, a Vysotski, E. S. (2014). Characterization of hydromedusan Ca(2+)-regulated photoproteins as a tool for measurement of Ca(2+) concentration. Analytical and Bioanalytical Chemistry, 406(23), 5715-26.
Malikova N P, Stepanyuk G A, Frank L A, Markova S V, Vysotski E S, Lee J. (2003) Spectral tuning of obelin bioluminescence by mutations of Trp92. FEBS Lett 554: 184-188.
Preston B. Rich and Christelle Douillet. Bioluminescence: Methods and Protocols (Methods in Molecular Biology) 2nd ed. 2009.
Rogers K L, Picaud S, Roncali E, Boisgard R, Colasante C, Stinnakre J, Tavitian B, Brulet P. 2007. Non-invasive in vivo imaging of Ca2+ signaling in mice. PLoS ONE 2:e974.
Sambrook et al. in “Molecular Cloning: A laboratory Manual”, 3^{rd} edition, Cold Spring Harbor Laboratory Press, (2001).
Shimomura O, Membrane permeability of coelenterazine analogues measured with fish eggs Biochem. J. (1997) 326, 297-298 Springer Protocols. Edition by Preston B. Rich, Christelle Douillet.
Stables, J., Green, A., Marshall, F., Fraser, N., Knight, E., Sautel, M., Milligan, G., Lee, M. and Rees, S. (1997). A bioluminescent assay for agonist activity at potentially any G-protein-coupled receptor. Anal. Biochem. 252, 115-126.
Stepanyuk, G. A., S. Golz, et al. (2005). “Interchange of aequorin and obelin bioluminescence color is determined by substitution of one active site residue of each photoprotein.” FEBS Lett 579(5): 1008-1014.
Tricoire L, Lambolez B. 2014. Neuronal network imaging in acute slices using Ca2+ sensitive bioluminescent reporter. Methods Mol Biol. 1098:33-45.
Tricoire, L., K. Tsuzuki, et al. (2006). “Calcium dependence of aequorin bioluminescence dissected by random mutagenesis.” Proc Natl Acad Sci USA 103(25): 9500-9505.
Tsuzuki K, et al (2005) Thermostable mutants of the photoprotein aequorin obtained by in vitro evolution. J Biol Chem. 280, 34324-31.
Ungrin M D, Singh L M R, Stocco R, Sas D E, Abramovitz M (1999) An automated aequorin luminescence-based functional calcium.

1-16. (canceled)

17. An apoaequorin mutant protein, wherein said apoaequorin mutant protein comprises an amino acid substitution in position 159, position 121, position 123, position 179, position 157 or in several of positions 159, 121, 123, 179 and 157 of reference wt-apoaequorin amino acid sequence as set forth in SEQ ID NO: 1.

18. The apoaequorin mutant protein according to claim 17, which comprises an amino acid substitution in position 159 of reference wt-apoaequorin amino acid sequence as set forth in SEQ ID NO: 1.

19. The apoaequorin mutant protein according to claim 18, which further comprises an amino acid substitution in position 121, position 123, position 179, position 157 or in several of positions 121, 123, 179 and 157 of reference wt-apoaequorin amino acid sequence as set forth in SEQ ID NO: 1.

20. The apoaequorin mutant protein according to claim 19, which comprises:
a) an amino acid substitution in position 159 and an amino acid substitution in position 121 of reference wt-apoaequorin amino acid sequence as set forth in SEQ ID NO: 1;
b) an amino acid substitution in position 159 and an amino acid substitution in position 123 of wild reference wt-apoaequorin amino acid sequence as set forth in SEQ ID NO: 1;
c) an amino acid substitution in position 159 and an amino acid substitution in position 179 of reference wt-apoaequorin amino acid sequence as set forth in SEQ ID NO: 1; or
d) an amino acid substitution in position 159 and an amino acid substitution in position 157 of reference wt-apoaequorin amino acid sequence as set forth in SEQ ID NO: 1.

21. The apoaequorin mutant protein according to claim 17, wherein:
a) the glutamine (Q) residue in position 159 of wt-apoaequorin amino acid sequence as set forth in SEQ ID NO: 1 is replaced by negatively-charged or not positively-charged hydrophilic amino acids;
b) the asparagine (N) residue in position 121 of reference wt-apoaequorin amino acid sequence as set forth in SEQ ID NO: 1 is replaced by negatively-charged amino acids;
c) the alanine (A) residue in position 123 of reference wt-apoaequorin amino acid sequence as set forth in SEQ ID NO: 1 is replaced by negatively-charged amino acids;
d) the alanine (A) residue in position 179 of reference wt-apoaequorin amino acid sequence as set forth in SEQ ID NO: 1 is replaced by not negatively-charged hydrophilic amino acids;
e) the serine (S) residue in position 157 of reference wt-apoaequorin amino acid sequence as set forth in SEQ ID NO: 1 is replaced by negatively-charged amino acids.

22. The apoaequorin mutant protein according to claim 17, wherein said apoaequorin mutant protein, when reconstituted with coelenterazine f (CLZ-f) cofactor, has an EC50 value for calcium of 492 nM or lower, and when reconstituted with native coelenterazine (CLZ) cofactor has an EC50 value for calcium of 680 nM or lower.

23. The apoaequorin mutant protein according to claim 17, which further comprises additional mutations compared to reference wt-apoaequorin amino acid sequence as set forth in SEQ ID NO: 1, in particular said additional mutations are selected from:
a) fusion with a fluorescent protein; and
b) amino acid substitutions that:
i) increase relative brightness of aequorin,
ii) increase decay kinetics of aequorin,
iii) increase thermostability of aequorin, and/or
iv) increase the wavelength of emission λ of aequorin.

24. The apoaequorin mutant protein according to claim 17, which is selected from the sequences as set forth in SEQ ID NO: 2 to SEQ ID NO: 25.

25. The apoaequorin mutant protein according to claim 17, which is further covalently conjugated to another molecule able to specifically bind a target molecule, preferentially the apoaequorin mutant protein is covalently conjugated to an antibody.

26. A nucleic acid molecule coding for the apoaequorin mutant protein according to claim 17.

27. A recombinant vector comprising the nucleic acid molecule coding for the apoaequorin mutant protein according to claim 17.

28. A host cell comprising the nucleic acid molecule coding for the apoaequorin mutant protein according to claim 17 or the recombinant vector comprising the nucleic acid molecule coding for the apoaequorin mutant protein according to claim 17.

29. A non-human transgenic animal comprising the nucleic acid molecule coding for the apoaequorin mutant protein according to claim 17 or the recombinant vector comprising the nucleic acid molecule coding for the apoaequorin mutant protein according to claim 17.

30. A kit comprising the apoaequorin mutant protein according to claim 17 and a wild-type or modified coelenterazine (CLZ) cofactor.

31. A kit comprising the apoaequorin mutant protein according to claim 18 and a wild-type or modified coelenterazine (CLZ) cofactor.

32. A reconstituted mutant aequorin complex, comprising the apoaequorin mutant protein according to claim 17, a native or modified coelenterazine (CLZ) cofactor, and molecular oxygen.

33. The apoaequorin mutant protein according to claim 21, wherein:
the glutamine (Q) residue in position 159 of reference wt-apoaequorin amino acid sequence as set forth in SEQ ID NO: 1 is replaced by an aspartic acid (D) residue, a threonine (T) residue, a glycine (G) residue or a glutamic acid (E) residue;
the asparagine (N) residue in position 121 of reference wt-apoaequorin amino acid sequence as set forth in SEQ ID NO: 1 is replaced by an aspartic acid (D) residue;
the alanine (A) residue in position 123 of reference wt-apoaequorin amino acid sequence as set forth in SEQ ID NO: 1 is replaced by an aspartic acid (D) residue;
the alanine (A) residue in position 179 of reference wt-apoaequorin amino acid sequence as set forth in SEQ ID NO: 1 is replaced by a threonine (T) residue; and
the serine (S) residue in position 157 of reference wt-apoaequorin amino acid sequence as set forth in SEQ ID NO: 1 is replaced by an aspartic acid (D) residue.

34. The apoaequorin mutant protein according to claim 22, wherein said apoaequorin mutant protein, when reconstituted with coelenterazine f (CLZ-f) cofactor, has an EC50 value for calcium of 216 nM or lower, and when reconstituted with native coelenterazine (CLZ) cofactor has an EC50 value for calcium of 470 nM or lower.

35. The apoaequorin mutant protein according to claim 23, wherein said fluorescent protein is selected from:
i) a green fluorescent protein (GFP),
ii) a citrine protein, or
iii) a tdTomato protein.

36. A kit comprising the apoaequorin mutant protein according to claim 19 and a wild-type or modified coelenterazine (CLZ) cofactor.


SEQUENCE LISTING (25 sequences)

SEQ ID NO: 1 - protein, 189 aa, Aequorea aequorea
  SITE (1)..(189): wild-type aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHLGFWYTMDPACEKLYGGAVP

SEQ ID NO: 2 - protein, 12 aa, Aequorea aequorea
  SITE (1)..(12): aequorin EF Hand
DVNHNGKISLDE

SEQ ID NO: 3 - protein, 12 aa, Aequorea aequorea
  SITE (1)..(12): aequorin EF Hand
DKDQNGAITLDE

SEQ ID NO: 4 - protein, 12 aa, Aequorea aequorea
  SITE (1)..(12): aequorin EF Hand
DIDESGQLDVDE

SEQ ID NO: 5 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGDLDVDEMTRQHLGFWYTMDPACEKLYGGAVP

SEQ ID NO: 6 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGTLDVDEMTRQHLGFWYTMDPACEKLYGGAVP

SEQ ID NO: 7 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGGLDVDEMTRQHLGFWYTMDPACEKLYGGAVP

SEQ ID NO: 8 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGELDVDEMTRQHLGFWYTMDPACEKLYGGAVP

SEQ ID NO: 9 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQDGAITLDEWKAYTKAAGIIQSSEDCEETFRVCDIDESGQLDVDEMTRQHLGFWYTMDPACEKLYGGAVP

SEQ ID NO: 10 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGDITLDEWKAYTKAAGIIQSSEDCEETFRVCDIDESGQLDVDEMTRQHLGFWYTMDPACEKLYGGAVP

SEQ ID NO: 11 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHLGFWYTMDPTCEKLYGGAVP

SEQ ID NO: 12 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDEDGQLDVDEMTRQHLGFWYTMDPACEKLYGGAVP

SEQ ID NO: 13 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQDGAITLDEWKAYTKAAGIIQSSEDCEETFRVCDIDESGDLDVDEMTRQHLGFWYTMDPACEKLYGGAVP

SEQ ID NO: 14 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQDGAITLDEWKAYTKAAGIIQSSEDCEETFRVCDIDESGTLDVDEMTRQHLGFWYTMDPACEKLYGGAVP

SEQ ID NO: 15 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGDITLDEWKAYTKAAGIIQSSEDCEETFRVCDIDESGDLDVDEMTRQHLGFWYTMDPACEKLYGGAVP

SEQ ID NO: 16 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGDITLDEWKAYTKAAGIIQSSEDCEETFRVCDIDESGTLDVDEMTRQHLGFWYTMDPACEKLYGGAVP

SEQ ID NO: 17 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGDLDVDEMTRQHLGFWYTMDPTCEKLYGGAVP

SEQ ID NO: 18 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGTLDVDEMTRQHLGFWYTMDPTCEKLYGGAVP

SEQ ID NO: 19 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDEDGDLDVDEMTRQHLGFWYTMDPACEKLYGGAVP

SEQ ID NO: 20 - protein, 189 aa, artificial
  mutant aequorin
SKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDEDGTLDVDEMTRQHLGFWYTMDPACEKLYGGAVP

SEQ ID NO: 21 - protein, 653 aa, artificial
  mutant aequorin
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLSGFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGDLDVDEMTRQHLGFWYTMDPACEKLYGGAVP

SEQ ID NO: 22 - protein, 653 aa, artificial
  mutant aequorin
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLSGFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGTLDVDEMTRQHLGFWYTMDPACEKLYGGAVP

SEQ ID NO: 23 - protein, 653 aa, artificial
  mutant aequorin
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLSGFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGDLDVDEMTRQHLGFWYTMDPTCEKLYGGAVP

SEQ ID NO: 24 - protein, 653 aa, artificial
  mutant aequorin
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLSGFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGTLDVDEMTRQHLGFWYTMDPTCEKLYGGAVP

SEQ ID NO: 25 - protein, 653 aa, artificial
  mutant aequorin
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLSGFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGDITLDEWKAYTKAAGIIQSSEDCEETFRVCDIDESGTLDVDEMTRQHLGFWYTMDPACEKLYGGAVP